(19) United States
(12) Patent Application Publication
(10) Pub. No.: US 2024/0199540 A1
(43) Pub. Date: Jun. 20, 2024

(54) CARBOXYLATE SALT, PHOTORESIST COMPOSITION INCLUDING THE SAME, AND METHOD OF FORMING PATTERN USING THE PHOTORESIST COMPOSITION

(71) Applicant: Samsung Electronics Co., Ltd., Suwon-si (KR)

(72) Inventors: Hoyoon PARK, Suwon-si (KR); Hana KIM, Suwon-si (KR); Kyuhyun IM, Suwon-si (KR); Haengdeog KOH, Suwon-si (KR); Yoonhyun KWAK, Suwon-si (KR); Hyeran KIM, Suwon-si (KR); Changheon LEE, Suwon-si (KR)

(73) Assignee: Samsung Electronics Co., Ltd., Suwon-si (KR)

(21) Appl. No.: 18/312,825

(22) Filed: May 5, 2023

(30) Foreign Application Priority Data
Nov. 30, 2022 (KR) 10-2022-0165094

(51) Int. Cl. C07C 323/09; G03F 7/004; G03F 7/20

(52) U.S. Cl. CPC C07C 323/09; G03F 7/0042; G03F 7/2004; G03F 7/2059; C07C 2601/16

(57) ABSTRACT

Provided are a carboxylate salt represented by Formula 1, a photoresist composition including the same, and a pattern forming method using the same:

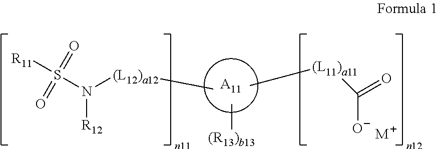

wherein A₁₁, L₁₁, L₁₂, a11, a12, R₁₁ to R₁₃, b13, n11, n12, and M⁺ in Formula 1 are defined as described in the specification.

### CROSS-REFERENCE TO RELATED APPLICATION

This application is based on and claims priority under 35 U.S.C. § 119 to Korean Patent Application No. 10-2022-0165094, filed on Nov. 30, 2022, in the Korean Intellectual Property Office, the disclosure of which is incorporated by reference herein in its entirety.

### BACKGROUND

### 1. Field

The disclosure relates to a carboxylate salt, a photoresist composition including the same, and a method of forming a pattern using the photoresist composition

### 2. Description of the Related Art

In semiconductor manufacturing, photoresists have physical properties that change in response to light and photoresists are used to form fine patterns. From among these photoresists, a chemically amplified photoresist has been widely used. In the case of chemically amplified photoresists, an acid formed by a reaction between light and a photoacid generator reacts again with a base resin to change the solubility of the base resin with respect to a developing solution, thereby enabling patterning.

In particular, in the case of using a high energy ray having relatively very high energy, such as EUV, the number of photons is remarkably small even when light having the same energy is irradiated. Accordingly, there may be a demand for a photoacid generator that can act effectively even when used in a small amount and can provide improved sensitivity and/or resolution.

In addition, in the case of chemically amplified photoresists, the formed acid diffuses to an unexposed area, and thus, the pattern uniformity may be reduced or the surface roughness may be increased. Accordingly, there may be a need for quenchers with improved dispersibility and/or improved compatibility with base resins.

### SUMMARY

Provided are a carboxylate salt, a photoresist composition including the same, and a pattern formation method using the photoresist composition, wherein the carboxylate salt may act as a photoacid generator capable of providing improved sensitivity and/or resolution and/or as a quencher having improved dispersibility and/or compatibility.

Additional aspects will be set forth in part in the description which follows and, in part, will be apparent from the description, or may be learned by practice of the presented embodiments of the disclosure.

According to an example embodiment, a carboxylate salt represented by Formula 1 is provided:

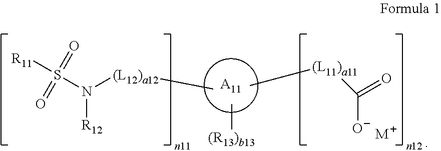

In Formula 1,
A₁₁ may be a C₁-C₃₀ cyclic hydrocarbon group that optionally includes a heteroatom,
L₁₁ and L₁₂ independently each may be a single bond or a divalent linking group,
a11 and a12 each independently may be an integer from 0 to 4,
R₁₁ to R₁₃ each independently may be hydrogen, deuterium, a halogen, a cyano group, a hydroxy group, a linear, branched, or cyclic C₁-C₂₀ monovalent hydrocarbon group that optionally includes a heteroatom, or —N(Q₁)(Q₂),
Q₁ and Q₂ each independently may be hydrogen, deuterium, a linear, branched, or cyclic C₁-C₂₀ monovalent hydrocarbon group that optionally includes a heteroatom,
b13 may be an integer from 0 to 10,
an adjacent two of R₁₁, R₁₂, R₁₃, L₁₁, and L₁₂ optionally may be bonded to each other to form a ring,
an adjacent two of a plurality of R₁₃ optionally may be bonded to each other to form a ring,
n11 and n12 each independently may be an integer from 1 to 10, and
M⁺ may be a counter cation.

According to an example embodiment, a photoresist composition may include the carboxylate salt, an organic solvent, and a base resin.

According to an example embodiment, a method of forming a pattern includes forming a photoresist film by applying the photoresist composition, exposing at least a portion of the photoresist film to high energy rays to provide an exposed photoresist film, and developing the exposed photoresist film by using a developer solution.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a flowchart illustrating a method of forming a pattern according to an embodiment;

FIGS. 2A to 2C are side cross-sectionals view illustrating a method of forming a pattern according to an embodiment;

FIG. 3 is a graph showing the degree of acid generation according to the exposure dose in Comparative Examples 1 and 2 and Example 1;

FIGS. 4A to 4E are side cross-sectional views illustrated a method of forming a patterned structure according to an embodiment; and

FIGS. 5A to 5E are side cross-sectional views illustrated a method of forming a semiconductor device according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figure, to explain aspects. As used herein, the term “and/or” includes any and all combinations of one or more of the associated listed items. Expressions such as “at least one of,” when preceding a list of elements, modify the entire list of elements and do not modify the individual elements of the list.

When the terms “about” or “substantially” are used in this specification in connection with a numerical value, it is intended that the associated numerical value includes a manufacturing or operational tolerance (e.g., ±10%) around the stated numerical value. Moreover, when the words “generally” and “substantially” are used in connection with geometric shapes, it is intended that precision of the geometric shape is not required but that latitude for the shape is within the scope of the disclosure. Further, regardless of whether numerical values or shapes are modified as “about” or “substantially,” it will be understood that these values and shapes should be construed as including a manufacturing or operational tolerance (e.g., ±10%) around the stated numerical values or shapes. When ranges are specified, the range includes all values therebetween such as increments of 0.1%.

Because the disclosure may have diverse modified embodiments, embodiments are illustrated in the drawings and are described in the detailed description. However, this is not intended to limit embodiments to particular modes of practice, and it is to be appreciated that all changes, equivalents, and substitutes that do not depart from the spirit and technical scope of the disclosure are encompassed in embodiments. In the description of embodiments certain detailed explanations of the related art are omitted when it is deemed that they may unnecessarily obscure the essence of the disclosure.

Terms such as “first”, “second”, “third”, and the like may be used to describe various components, but are used only for the purpose of distinguishing one component from other components, and the order, type, and the like of the components are not limited.

It will be understood that when a component, such as a layer, a film, a region, or a plate, is referred to as being “on” or “above” another component in the specification, the component can directly contact to be above, below, right, or left of the another component as well as being above, below, left, or right of the another component in a non-contact manner.

An expression used in the singular encompasses the expression of the plural, unless it has a clearly different meaning in the context. It is to be understood that the terms such as “including,” “having,” and “comprising” are intended to indicate the existence of the features, numbers, steps, actions, components, parts, ingredients, materials, or combinations thereof disclosed in the specification, and are not intended to preclude the possibility that one or more other features, numbers, steps, actions, components, parts, ingredients, materials, or combinations thereof may exist or may be added.

Whenever a range of values is enumerated, the range includes all values within the range as if recorded explicitly clearly, and may further include the boundaries of the range. Accordingly, the expression in a range of “X” to “Y” includes all values between X and Y, including X and Y.

The term “Cₓ-C_{y}” used herein refers to a case where the number of carbons constituting the substituent is x to y. For example, “C₁-C₆” refers to a case where the number of carbons constituting the substituent is 1 to 6, and “C₆-C₂₀” refers to a case where the number of carbons constituting the substituent is 6 to 20.

The term “monovalent hydrocarbon group” used herein may include, for example, a linear or branched alkyl group (for example, a methyl group, an ethyl group, a propyl group, an isopropyl group, a butyl group, an isobutyl group, a sec-butyl group, a tert-butyl group, a pentyl group, a neopentyl group, a hexyl group, a heptyl group, a 2-ethylhexyl group, and a nonyl group); a monovalent saturated cycloaliphatic hydrocarbon group (for example, a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a 1-adamantyl group, a 2-adamantyl group, a 1-adamantylmethyl group, a norbornyl group, a norbornylmethyl group, a tricyclodecanyl group, a tetracyclododecanyl group, a tetracyclododecanylmethyl group, and a dicyclohexylmethyl group); a monovalent unsaturated aliphatic hydrocarbon group (for example, an allyl group and a 3-cyclohexenyl group); an aryl group (for example, a phenyl group, a 1-naphthyl group, and a 2-naphthyl group); an arylalkyl group (for example, a benzyl group and a diphenylmethyl group); and a monovalent hydrocarbon group containing a heteroatom (for example, a tetrahydrofuranyl group, a methoxymethyl group, an ethoxymethyl group, and a methylthiomethyl group, an acetamidemethyl group, a trifluoroethyl group, a (2-methoxyethoxy)methyl group, an acetoxymethyl group, a 2-carboxy-1-cyclohexyl group, a 2-oxopropyl group, a 4-oxo-1-adamantyl group, and a 3-oxocyclohexyl group). In addition, among these groups, some hydrogen atoms may be substituted with a moiety including a heteroatom, such as oxygen, sulfur, nitrogen, or a halogen atom, or some carbon atoms may be replaced by a moiety including a heteroatom, such as oxygen, sulfur, or nitrogen. Accordingly, these groups may each include a hydroxy group, a cyano group, a carbonyl group, a carboxyl group, an ether linking group, an ester linking group, a sulfonic ester linking group, a carbonate, a lactone ring, a sultone ring, a carboxylic anhydride moiety, or a haloalkyl moiety.

The term “alkyl group” used herein refers to a linear or branched saturated aliphatic hydrocarbon monovalent group, and examples thereof include a methyl group, an ethyl group, a propyl group, an isobutyl group, a sec-butyl group, a tert-butyl group, a pentyl group, an iso-amyl group, a hexyl group, and the like. The term “alkylene group” used herein refers to a linear or branched saturated aliphatic hydrocarbon divalent group, and examples thereof include a methylene group, an ethylene group, a propylene group, a butylene group, an isobutyl group, and the like.

The term “halogenated alkyl group” used herein refers to a group in which one or more substituents of an alkyl group are substituted with halogen, and examples include CF₃ and the like. Here, a halogen is F, Cl, Br, or I.

The term “alkoxy group” used herein refers to a monovalent group represented by —OA₁₀₁, where A₁₀₁ is an alkyl group. Examples thereof include a methoxy group, an ethoxy group, an isopropyloxy group, and the like.

The term “cycloalkyl group” used herein refers to a monovalent saturated hydrocarbon cyclic group, and examples thereof include monocyclic groups such as a cyclopropyl group, a cyclobutyl group, a cyclopentyl group, a cyclohexyl group, a cycloheptyl group, and the like, and polycyclic condensed cyclic groups such as a norbornyl group and an adamantyl group. The term “cycloalkylene group” used herein refers to a divalent saturated hydrocarbon cyclic group, and examples thereof include a cyclopentylene group, a cyclohexylene group, an adamantylene group, an adamantylmethylene group, a norbornylene group, a norbornylmethylene group, a tricyclodecanylene group, a tetracyclododecanylene group, a tetracyclododecanylmethylene group, a dicyclohexylmethylene group, and the like.

The term “cycloalkoxy group” used herein refers to a monovalent group represented by —OA₁₀₂, where A₁₀₂ is a cycloalkyl group. Examples thereof include a cyclopropoxy group, a cyclobutoxy group, and the like.

The term “heterocycloalkyl group” used herein refers to a group in which some carbon atoms of the cycloalkyl group are replaced by a moiety including a heteroatom, such as oxygen, sulfur, or nitrogen, and examples of the heterocycloalkyl group include an ether linking group, an ester linking group, a sulfonic ester linking group, a carbonate, a lactone ring, a sultone ring, or a carboxylic anhydride moiety. The term “heterocycloalkylene group” used herein refers to a group in which some carbon atoms of the cycloalkylene group are replaced by a moiety including a heteroatom, such as oxygen, sulfur, or nitrogen.

The term “alkenylene group” used herein refers to a linear or branched unsaturated divalent aliphatic hydrocarbon group.

The term “cycloalkenylene group” used herein refers to an unsaturated divalent hydrocarbon cyclic group.

The term “heterocycloalkenylene group” used herein refers to a group in which some carbon atoms of the cycloalkenylene group are replaced by a moiety including a heteroatom, such as oxygen, sulfur, or nitrogen.

The term “aryl group” used herein refers to a monovalent group having a carbocyclic aromatic system, and examples thereof include a phenyl group, a naphthyl group, an anthracenyl group, a phenanthrenyl group, a pyrenyl group, a chrysenyl group, and the like. The term “arylene group” used herein refers to a divalent group having a carbocyclic aromatic system.

The term “heteroaryl group” used herein refers to a monovalent group having a heterocyclic aromatic system, and examples thereof include a pyridinyl group, a pyrimidinyl group, a pyrazinyl group, and the like. The term “heteroarylene group” used herein refers to a divalent group having a heterocyclic aromatic system.

The term “cycloalkyl group” used herein refers to a monovalent, divalent, or trivalent group in which any hydrogen of the cycloalkyl group can be further substituted as a binding site.

The term “heterocycloalkyl group” used herein refers to a monovalent, divalent, or trivalent group in which any hydrogen of the heterocycloalkyl group can be further substituted as a binding site.

The term “cycloalkenyl group” used herein refers to a monovalent, divalent, or trivalent group in which any hydrogen of the cycloalkenyl group can be further substituted as a binding site.

The term “heterocycloalkenyl group” used herein refers to a monovalent, divalent, or trivalent group in which any hydrogen of the heterocycloalkenyl group can be further substituted as a binding site.

The term “aryl group” used herein refers to a monovalent, divalent, or trivalent group in which any hydrogen of the aryl group can be further substituted as a binding site.

The term “heteroaryl group” used herein refers to a monovalent, divalent, or trivalent group in which any hydrogen of the heteroaryl group can be further substituted as a binding site.

The term “counter cation” used herein refers to any cation that can be ionically bonded with an anion, and generally refers to a cation that prevents a compound from being charged.

Hereinafter, the disclosure will be described in detail by explaining embodiments with reference to the accompanying drawings, and substantially identical or corresponding components are given the same reference numerals in the drawings, and thus a description thereof will be omitted. Regarding the drawings, the thickness is shown enlarged to clearly express the various layers and regions. Also, in the drawings, the thicknesses of some layers and regions are exaggerated for convenience of description. Meanwhile, embodiments described below are illustrative examples of embodiments, and various changes in forms and details may be made.

### Carboxylate Salt

A carboxylate salt according to one or more embodiments may be represented by Formula 1:

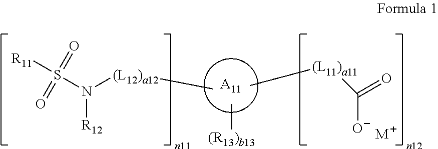

In Formula 1,
A₁₁ may be a C₁-C₃₀ cyclic hydrocarbon group that optionally includes a heteroatom,
L₁₁ and L₁₂ independently each may be a single bond or a divalent linking group,
a11 and a12 each independently may be an integer from 0 to 4,
R₁₁ to R₁₃ each independently may be hydrogen, deuterium, a halogen, a cyano group, a hydroxy group, a linear, branched, or cyclic C₁-C₂₀ monovalent hydrocarbon group that optionally includes a heteroatom, or —N(Q₁)(Q₂),
Q₁ and Q₂ each independently may be hydrogen, deuterium, a linear, branched, or cyclic C₁-C₂₀ monovalent hydrocarbon group that optionally includes a heteroatom,
b13 may be an integer from 0 to 10,
an adjacent two of R₁₁, R₁₂, R₁₃, L₁₁, and L₁₂ optionally may be bonded to each other to form a ring,
an adjacent two of a plurality of R₁₃ optionally may be bonded to each other to form a ring,
n11 and n12 each independently may be an integer from 1 to 10, and
M⁺ may be a counter cation.

For example, A₁₁ in Formula 1 may be a C₃-C₂₀ cycloalkyl group, a C₃-C₂₀ heterocycloalkyl group, a C₃-C₂₀ cycloalkenyl group, a C₃-C₂₀ heterocycloalkenyl group, a C₆-C₂₀ aryl group, or a C₁-C₂₀ heteroaryl group.

In detail, A₁₁ in Formula 1 may be a cyclopentyl group, a cyclohexyl group, a tetrahydrofuran group, a tetrahydropyran group, a norbornyl group, a tricyclodecanyl group, a tetracyclododecanyl group, an adamantyl group, an oxa-norbornyl group, an oxa-tricyclodecanyl group, an oxa-tetracyclododecanyl group, an oxa-adamantyl group, a benzene group, a naphthalene group, a phenanthrene group, an anthracene group, a furan group, or a thiophene group.

In more detail, A₁₁ in Formula 1 may be selected from Formulae 3-1 to 3-6:

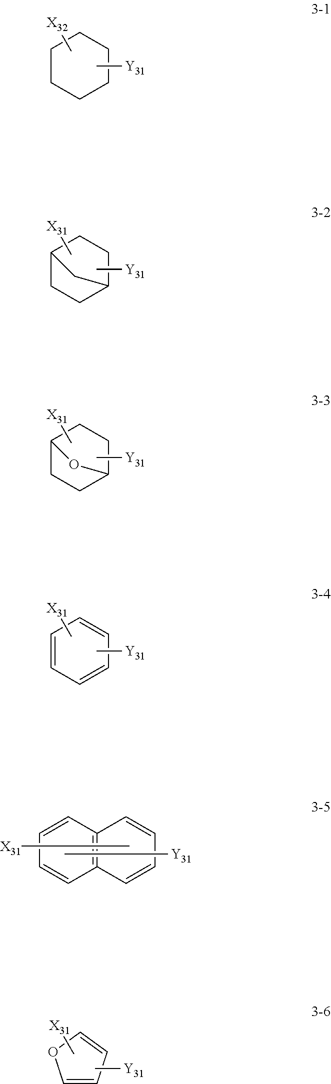

In Formulae 3-1 to 3-6,
X₃₁ may be represented by

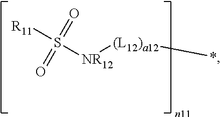

and
Y₃₁ may be represented by

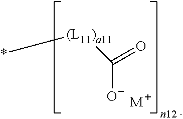

For example, L₁₁ and L₁₂ in Formula 1 may each independently be a single bond, O, C(═O), C(═O)O, OC(═O), C(═O)NH, NHC(═O), or a linear, branched, or cyclic C₁-C₃₀ divalent group that optionally includes a heteroatom.

In more detail, L₁₁ and L₁₂ in Formula 1 may each independently be a single bond, O, C(═O), C(═O)O, OC(═O), C(═O)NH, NHC(═O), a substituted or unsubstituted C₁-C₃₀ alkylene group, a substituted or unsubstituted C₃-C₃₀ cycloalkylene group, a substituted or unsubstituted C₃-C₃₀ heterocycloalkylene group, a substituted or unsubstituted C₂-C₃₀ alkenylene group, a substituted or unsubstituted C₃-C₃₀ cycloalkenylene group, a substituted or unsubstituted C₃-C₃₀ heterocycloalkenyl group, a substituted or unsubstituted C₆-C₃₀ arylene group, or a substituted or unsubstituted C₁-C₃₀ heteroarylene group.

In more detail, L₁₁ and L₁₂ in Formula 1 may each independently be selected from: a single bond; C(═O); C(═O)O; OC(═O); and a C₁-C₃₀ alkylene group, a C₃-C₃₀ cycloalkylene group, a C₃-C₃₀ heterocycloalkylene group, a C₂-C₃₀ alkenylene group, a C₃-C₃₀ cycloalkenylene group, a C₃-C₃₀ heterocycloalkenylene group, a C₆-C₃₀ arylene group, and a C₁-C₃₀ heteroarylene group, each unsubstituted or substituted with deuterium, a halogen, a cyano group, a hydroxy group, an amino group, a carboxylic acid group, an ester moiety, a sulfonic ester moiety, a carbonate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₃-C₂₀ cycloalkoxy group, a C₆-C₂₀ aryl group, or any combination thereof.

In particular, L₁₁ and L₁₂ in Formula 1 may each independently be selected from: a single bond; C(═O); C(═O)O; OC(═O); and Formulae 9-1 to 9-3:

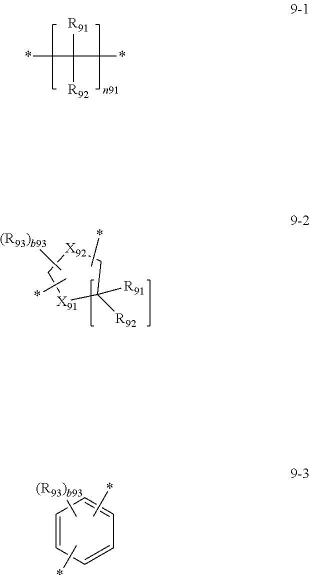

In Formulae 9-1 to 9-3,
X₉₁ may be O, S, or CR₉₄R₉₅,
X₉₂ may be O, S, or CR₉₆R₉₇,
R₉₁ to R₉₇ may each independently be selected from hydrogen, deuterium, a halogen , a cyano group, a hydroxy group, an amino group, a carboxylic acid group, a C₁-C₂₀ alkyl group, a C₁-C₂₀ halogenated alkyl group, a C₁-C₂₀ alkoxy group, a C₃-C₂₀ cycloalkyl group, a C₃-C₂₀ cycloalkoxy group, and a C₆-C₂₀ aryl group, and may optionally be bonded to form a ring,
b93 may be an integer from 1 to 4,
n91 may be an integer from 1 to 4, and
* and *′ each indicate a binding site to a neighboring atom.

In an embodiment, in Formula 1, L₁₁ may be a single bond, and L₁₂ may be selected from: a single bond; C(═O); C(═O)O; OC(═O); and Formulae 9-1 to 9-3.

In Formula 1, a11 indicates the number of repetitions of L₁₁. In Formula 1, when a11 is 0, (L₁₁)ₐ₁₁ is a single bond. In Formula 1, a12 indicates the number of repetitions of L₁₂. In Formula 1, when a12 is 0, (L₁₂)ₐ₁₂ is a single bond.

In an embodiment, in Formula 1, a11 may be 0, and a12 may be an integer from 0 to 4.

In one or more embodiments, a11 and a12 in Formula 1 may each be 0.

For example, in Formula 1, R₁₁ to R₁₃ may each independently be: hydrogen; deuterium; a halogen; a cyano group; a hydroxy group; —N(Q₁)(Q₂); and a C₁-C₂₀ alkyl group, a C₃-C₂₀ cycloalkyl group, and a C₆-C₂₀ aryl group, each unsubstituted or substituted with deuterium, a halogen, a cyano group, a hydroxy group, an amino group, a carboxylic acid group, an ester moiety, a sulfonic ester moiety, a carbonate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₃-C₂₀ cycloalkoxy group, a C₆-C₂₀ aryl group, a C₁-C₂₀ alkylamino group, a C₁-C₂₀ arylamino group, or any combination thereof, and
Q₁ and Q₂ may each independently be selected from: hydrogen; deuterium; and a C₁-C₂₀ alkyl group, a C₃-C₂₀ cycloalkyl group, and a C₆-C₂₀ aryl group, each unsubstituted or substituted with deuterium, a halogen, a cyano group, a hydroxy group, an amino group, a carboxylic acid group, an ester moiety, a sulfonic ester moiety, a carbonate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₃-C₂₀ cycloalkoxy group, a C₆-C₂₀ aryl group, a C₁-C₂₀ alkylamino group, a C₁-C₂₀ arylamino group, or any combination thereof.

In detail, R₁₁ to R₁₃ in Formula 1 may each independently be selected from: hydrogen; deuterium; a halogen; a cyano group; a hydroxy group; and a C₁-C₂₀ alkyl group, a C₃-C₂₀ cycloalkyl group, and a C₆-C₂₀ aryl group, each unsubstituted or substituted with deuterium, a halogen, a C₁-C₂₀ alkyl group, a C₁-C₂₀ halogenated alkyl group, a C₃-C₂₀ cycloalkyl group, a C₆-C₂₀ aryl group, or any combination thereof.

In an embodiment, R₁₁ in Formula 1 may not include a heteroatom. In detail, R₁₁ in Formula 1 may be selected from: hydrogen; deuterium; a C₁-C₂₀ alkyl group; a C₃-C₂₀ cycloalkyl group; and a C₆-C₂₀ aryl group.

In an embodiment, R₁₂ in Formula 1 may be hydrogen or deuterium.

In an embodiment, R₁₁ and L₁₂; R₁₁ and R₁₂; R₁₂ and L₁₂; or R₁₁ and R₁₃ may be bonded to each other to form a 5-membered ring or a 6-membered ring.

In an embodiment, when b13 is 2 or more, adjacent two R₁₃(s) may be bonded to each other to form a 5-membered ring or a 6-membered ring.

For example, n11 and n12 in Formula 1 may each independently be an integer from 1 to 3.

In detail, n11 and n12 in Formula 1 may each independently be 1 or 2.

For example, the sum of n11 and n12 in Formula 1 may be an integer from 2 to 5.

In detail, the sum of n11 and n12 in Formula 1 may be 2 or 3.

In an embodiment, in Formula 1, n11 may be 1 or 2, and n12 may be 1.

In one or more embodiments, n11 and n12 in Formula 1 may each be 1.

For example, M⁺ in Formula 1 may be a substituted or unsubstituted sulfonium cation, a substituted or unsubstituted iodonium cation, or a substituted or unsubstituted ammonium cation.

In detail, M⁺ in Formula 1 may be represented by any one of Formulae 2-1 to 2-3:

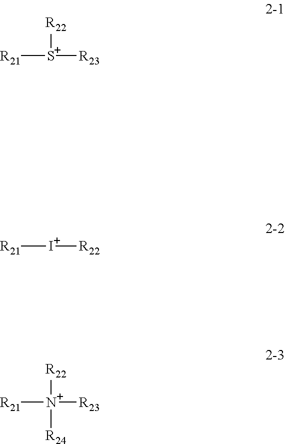

In Formulae 2-1 to 2-3,
R₂₁ to R₂₄ may each independently be a linear, branched, or cyclic C₁-C₃₀ monovalent hydrocarbon group that optionally includes a heteroatom, and
adjacent two of R₂₁ to R₂₄ may optionally be bonded to each other to form a ring.

In detail, M⁺ in Formula 1 may be represented by any one of Formulae 2-11 to 2-13:

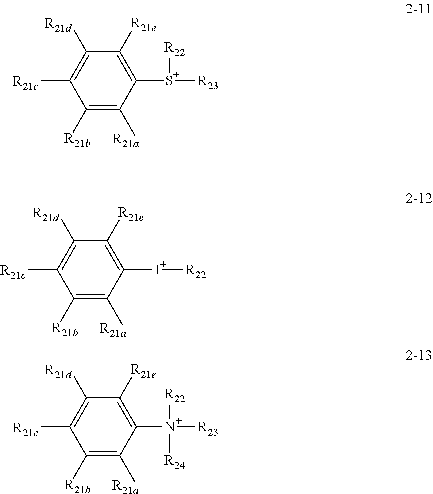

In Formulae 2-11 to 2-13,
R₂₁ₐ to R₂₁ₑ may each independently be: hydrogen; deuterium; a halogen; a cyano group; a hydroxy group; or a linear, branched, or cyclic C₁-C₂₀ monovalent hydrocarbon group that optionally includes a heteroatom,
R₂₂ to R₂₄ may each independently be a linear, branched, or cyclic C₁-C₃₀ monovalent hydrocarbon group that optionally includes a heteroatom, and
an adjacent two of R₂₁ₐ to R₂₁ₑ and R₂₂ to R₂₄ may optionally be bonded to each other to form a ring.

In particular, M⁺ in Formula 1 may be represented by any one of Formulae 2-21 to 2-23:

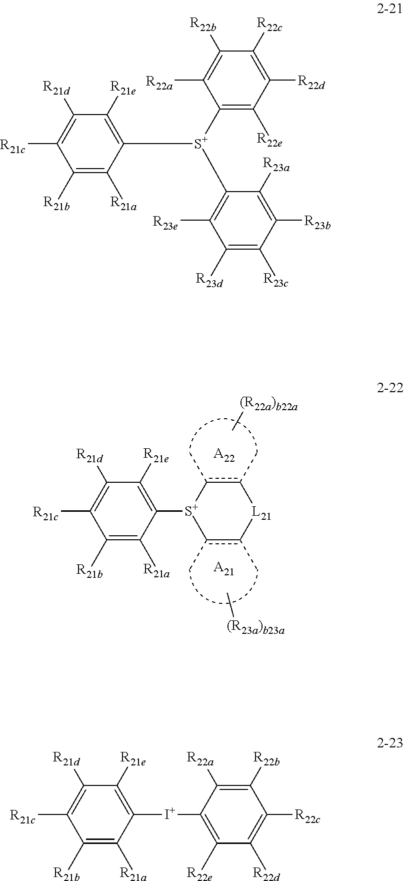

In Formulae 2-21 to 2-23,
R₂₁ₐ to R₂₁ₑ, R₂₂ₐ to R₂₂ₑ, and R₂₃ₐ to R₂₃ₑ may each independently be: hydrogen; deuterium; a halogen; a cyano group; a hydroxy group; or a linear, branched, or cyclic C₁-C₂₀ hydrocarbon group that optionally includes a heteroatom,
an adjacent two of R₂₁ₐ to R₂₁ₑ, R₂₂ₐ to R₂₂ₑ, and R₂₃ₐ to R₂₃ₑ may optionally be linked to form a condensed ring,
b22a and b23a may each be an integer from 1 to 4,
A₂₁ and A₂₂ may each independently be absent or a benzene ring,
the respective [symbol] may be a carbon-carbon single bond or a carbon-carbon double bond,
L₂₁ may be a single bond, O, S, CO, SO, SO₂, CRR′, or NR, and
R and R′ may each independently be: hydrogen; deuterium; a halogen; a cyano group; a hydroxy group; or a linear, branched, or cyclic C₁-C₂₀ hydrocarbon group that optionally includes a heteroatom.

In an embodiment, M⁺ in Formula 1 may be selected from Group II:

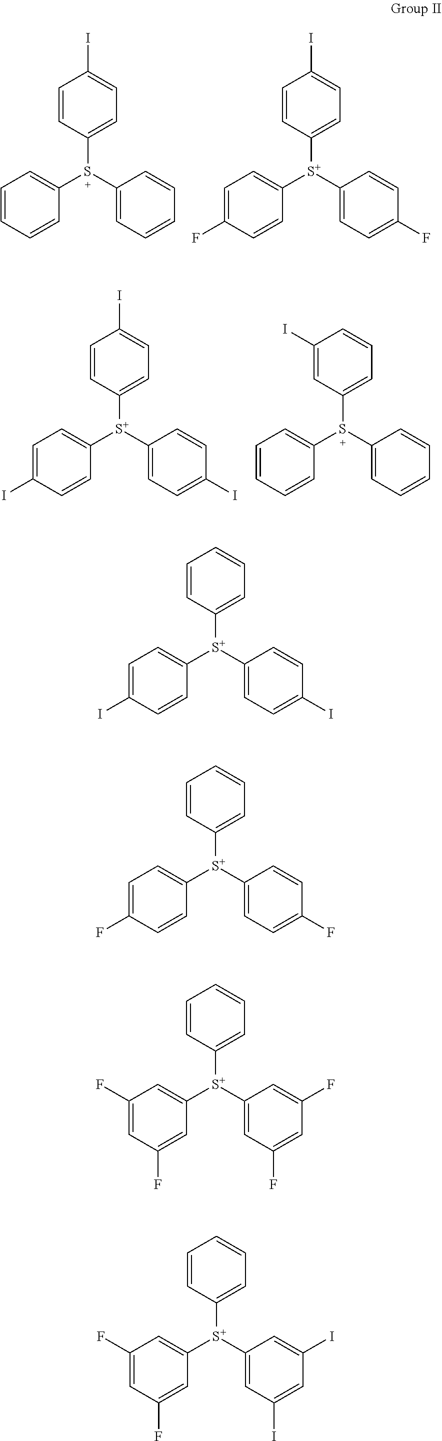

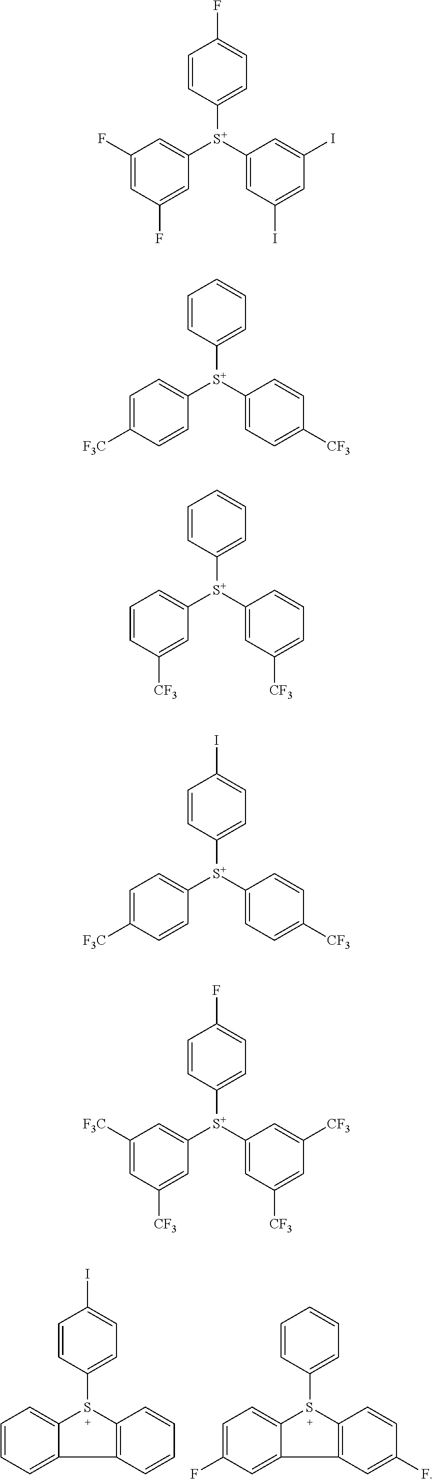

In an embodiment, the carboxylate salt represented by Formula 1 may be represented by any one of Formulae 1-1 to 1-3:

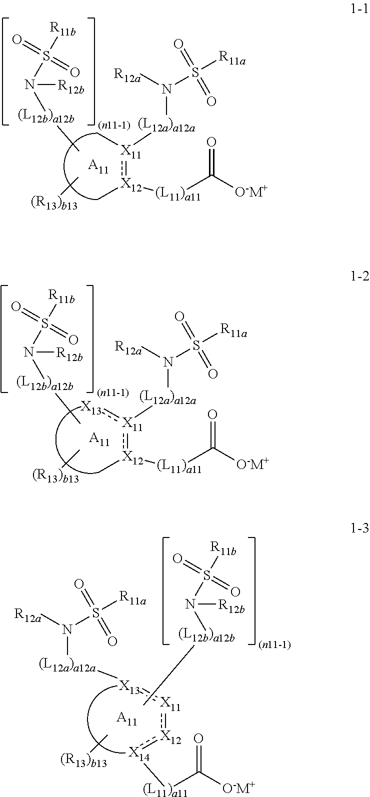

In Formulae 1-1 to 1-3,
X₁₁ to X₁₄ may each be any atom (e.g., C, O, N, S),
the respective [symbol] indicates a single bond or a double bond,
A₁₁, L₁₁, a11, M⁺, R₁₃, n11, and b13 are each defined the same as described above,
R₁₁ₐ and R_{11b} are each defined the same as R₁₁,
R₁₂ₐ and R_{12b} are each defined the same as R₁₂,
L₁₂ₐ and L_{12b} are each defined the same as L₁₂, and
a12a and a12b are each defined the same as a12.

In one or more embodiments, the carboxylate salt represented by Formula 1 may be represented by any one of Formulae 1-11 to 1-15:

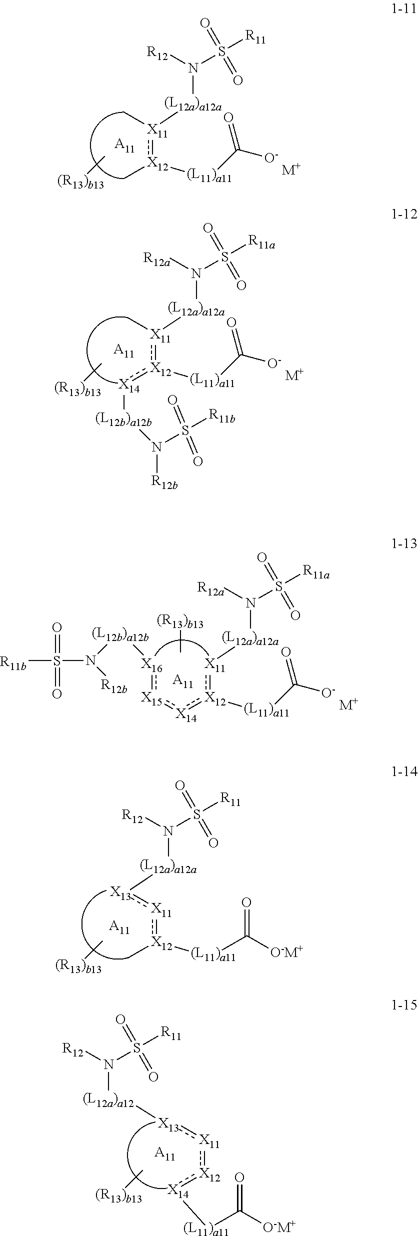

In Formulae 1-11 to 1-15,
X₁₁ to X₁₆ may each be any atom (e.g., C, O, N, S),
the respective [symbol] indicates a single bond or a double bond,
A₁₁, L₁₁, L₁₂, a11, a12, M⁺, R₁₁ to R₁₃, n11, n12, and b13 are defined as described above,
R₁₁ₐ and R_{11b} are each defined the same as R₁₁,
R₁₂ₐ and R_{12b} are each defined the same as R₁₂,
L₁₂ₐ and L_{12b} are each defined the same as L₁₂, and
a12a and a12b are each defined the same as a12.

In particular, the carboxylate salt represented by Formula 1 may be represented by Formula 1-1 or 1-2.

In detail, the carboxylate salt represented by Formula 1 may be selected from Group I:

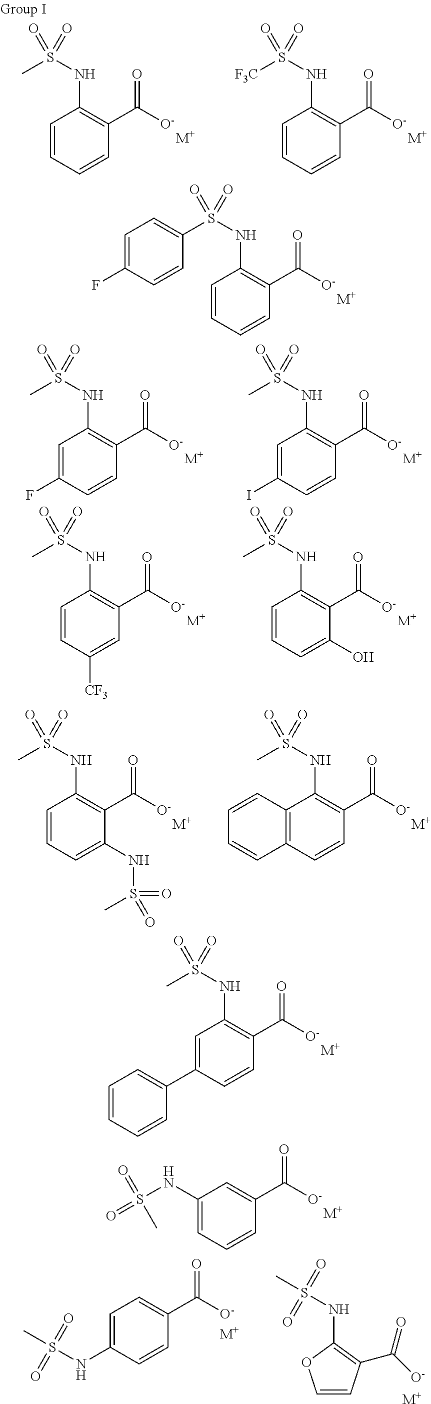

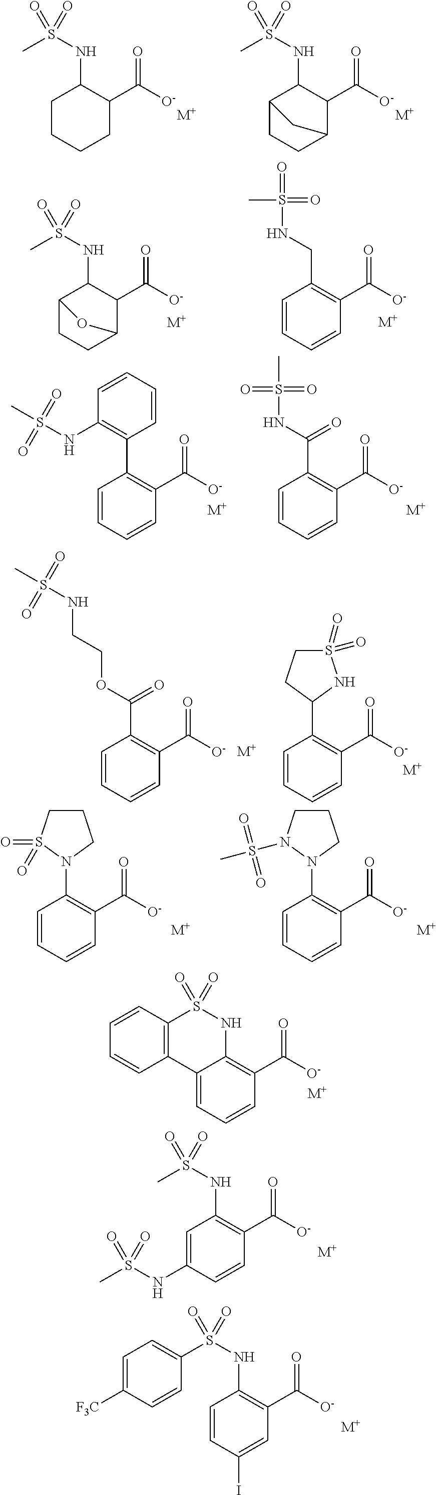

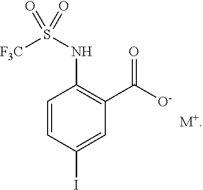

In Group I,
M⁺ may be a counter cation.

In detail, M⁺ in Group I may be selected from Group II.

In general, when forming a pattern by using a photoresist composition, an acid generated from a photoacid generator by light exposure may diffuse in a photoresist film. Accordingly, the acid may penetrate even to an unexposed area so that the sensitivity and/or resolution of the photoresist composition may be lowered. That is, to improve the sensitivity and/or resolution of the photoresist composition, the diffusion of the acid may be effectively reduced, and a quencher may be used for this purpose.

However, the use of a quencher may not simply reduce the diffusion of the acid. To enhance the effect of the quencher while using the quencher in an appropriate amount, there is a need to improve the dispersion degree of the quencher and/or the compatibility of the quencher with a base resin.

In particular, to improve the compatibility of the quencher with a base resin, a method of coupling macromolecules has been studied, but such a method could not solve the problems of a decrease in the solubility of the quencher in an organic solvent and/or a decrease in the compatibility of the quencher with a base resin. In addition, to improve the compatibility of the quencher with a base resin, a method of binding the quencher to a base resin itself has been studied, but practical applications had many difficulties due to a decrease in the solubility of the base resin itself and/or the influence of the quencher on the contrast.

In addition, when a low-molecular quencher is used, aggregation occurs in the base resin due to interactions among molecules of the low-molecular quencher (in particular, interactions by electrostatic attraction among ion-binding molecules). Also, a small amount of the quencher is not enough to effectively reduce the diffusion of the acid.

However, when the carboxylate salt represented by Formula 1 is used as the quencher, due to increased attraction between heterogeneous molecules compared to attraction between homogeneous molecules, the dispersibility of the acid in the base resin may be improved, and thus the diffusion of the acid may be effectively reduced even with a small amount of the quencher.

In addition, generally due to the difference in the diffusion distance of the acid, the roughness of the surface of the photoresist film may be increased after development. In this regard, when a quencher having the carboxylate salt according to an embodiment is used, the diffusion of the acid may be effectively and evenly reduced, thereby improving the surface roughness.

### Photoresist Composition

According to another aspect of the disclosure, a photoresist composition includes the carboxylate salt, an organic solvent, and a base resin. The photoresist composition may have properties including improved developability and/or improved resolution.

The solubility of the photoresist composition in a developing solution may be changed by exposure with high-energy rays. The photoresist composition may be: a positive photoresist composition that forms a positive photoresist pattern after an exposed area of a photoresist film is dissolved and removed; or a negative photoresist composition that forms a negative photoresist pattern after an unexposed area of a photoresist film is dissolved and removed. In addition, a sensitive photoresist composition according to an embodiment may be for an alkali development process using an alkali developing solution for the development treatment when forming a photoresist pattern formation, or may be for a solvent development process using a developing solution containing an organic solvent (hereinafter also referred to as an organic developing solution) for the development treatment.

The carboxylate salt may be a photo-decomposable compound that is decomposable by light exposure. The carboxylate salt may act as a photoacid generator since an acid is generated when decomposed by exposure, and thus, the photoresist composition may not include a separate photoacid generator. Instead, the photoresist composition may further include a quencher. In an embodiment, the carboxylate salt may be a photodegradable compound which may generate an acid by exposure and, before the exposure, acts as a quenching base to neutralize the acid. Here, the carboxylate salt may be used in combination with a photoacid generator that generates an acid. In addition, since the carboxylate salt may generate an acid upon light exposure, quencher functions of the carboxylate salt may be lost by neutralization with the acid generated by the carboxylate salt itself, and thus the contrast between the exposed are and the unexposed area may be further enhanced.

The carboxylate salt may be used in an amount in a range of 0.1 parts by weight to 40 parts by weight, for example, 5 parts by weight to 30 parts by weight, based on 100 parts by weight of the base resin. Within these ranges, the photoacid generator and/or quencher may show at appropriate levels of functions, and any performance loss, for example, the formation of foreign particles due to a decrease in sensitivity and/or lack of solubility may be reduced.

Since the carboxylate salt is as described above, hereinafter, an organic solvent, a base resin, and a photoacid generator, which is an optional component, will be described below. In addition, for use as the carboxylate salt represented by Formula 1 in the photoresist composition, one type of the carboxylate salt or a combination of two or more different types of the carboxylate salt may be used.

### Organic Solvent

The organic solvent included in the photoresist composition may not be particularly limited as long as it is capable of dissolving or dispersing the carboxylate salt, the base resin, the photoacid generator, and optional components contained as necessary. One type of the organic solvent may be used, or a combination of two or more different types of the organic solvent may be used. Also, a mixed solvent in which water and an organic solvent are mixed may be used.

Examples of the organic solvent are an alcohol-based solvent, an ether-based solvent, a ketone-based solvent, an amide-based solvent, an ester-based solvent, a sulfoxide-based solvent, a hydrocarbon-based solvent, and the like.

In detail, examples of the alcohol-based solvent are: a monoalcohol-based solvent, such as methanol, ethanol, n-propanol, isopropanol, 1-methoxy-2-propanol, 1-ethoxy-2-propanol, n-butanol, isobutanol, sec-butanol, tert-butanol, n-pentanol, isopentanol, 2-methylbutanol, sec-pentanol, tert-pentanol, 3-methoxybutanol, 3-methyl-3-methoxybutanol, n-hexanol, 2-methylpentanol, sec-hexanol, 2-ethylbutanol, sec-heptanol, 3-heptanol, n-octanol, 2-ethylhexanol, sec-octanol, n-nonyl alcohol, 2,6-dimethyl-4-heptanol, n-decanol, sec-undecyl alcohol, trimethylnonyl alcohol, sec-tetradecyl alcohol, sec-heptadecyl alcohol, furfuryl alcohol, phenol, cyclohexanol, methylcyclohexanol, 3,3,5-trimethylcyclohexanol, benzyl alcohol, diacetone alcohol, and the like; a polyhydric alcohol-based solvent, such as ethylene glycol, 1,2-propylene glycol, 1,3-butylene glycol, 2,4-pentanediol, 2-methyl-2,4-pentanediol, 2,5-hexanediol, 2,4-heptanediol, 2-ethyl-1,3-hexanediol, diethylene glycol, dipropylene glycol, triethylene glycol, tripropylene glycol, and the like; a polyhydric alcohol-containing ether-based solvent, such as ethyleneglycol monomethyl ether, ethylene glycol monoethyl ether, ethylene glycol monopropyl ether, ethylene glycol monobutyl ether, ethylene glycol monohexyl ether, ethylene glycol monophenyl ether, ethylene glycol mono-2-ethylbutyl ether, diethylene glycol monomethyl ether, diethylene glycol monoethyl ether, diethylene glycol monopropyl ether, diethylene glycol monobutyl ether, diethylene glycol monohexyl ether, diethylene glycol dimethyl ether, propylene glycol monomethyl ether, propylene glycol dimethyl ether, propylene glycol monoethyl ether, propylene glycol monopropyl ether, propylene glycol monobutyl ether, dipropylene glycol monomethyl ether, dipropylene glycol monoethyl ether, dipropylene glycol monopropyl ether, and the like; and the like.

Examples of the ether-based solvent are: a dialkyl ether-based solvent, such as diethyl ether, dipropyl ether, dibutyl ether, and the like; a cyclic ether-based solvent, such as tetrahydrofuran, tetrahydropyran, and the like; an aromatic ring-containing ether-based solvent, such as diphenyl ether, anisole, and the like; and the like.

Examples of the ketone-based solvent are: a chain ketone-based solvent, such as acetone, methylethyl ketone, methyl-n-propyl ketone, methyl-n-butyl ketone, methyl-n-pentyl ketone, diethyl ketone, methyl isobutyl ketone, 2-heptanone, ethyl-n-butyl ketone, methyl-n-hexyl ketone, diisobutyl ketone, trimethylnonanone, and the like; a cyclic ketone solvent, such as cyclopentanone, cyclohexanone, cycloheptanone, cyclooctanone, methylcyclohexanone, and the like; 2,4-pentanedione; acetonylacetone; acetphenone; and the like.

Examples of the amide-based solvent are: a cyclic amide-based solvent, such as N,N′-dimethylimidazolidinone, N-methyl-2-pyrrolidone, and the like; a chain amide-based solvent, such as N-methylformamide, N,N-dimethylformamide, N,N-diethylformamide, acetamide, N-methylacetamide, N,N-dimethylacetamide, N-methylpropionamide, and the like; and the like.

Examples of the ester-based solvent are: an acetate ester-based solvent, such as methyl acetate, ethyl acetate, n-propyl acetate, isopropyl acetate, n-butyl acetate, isobutyl acetate, sec-butyl acetate, t-butyl acetate, n-pentyl acetate, isopentyl acetate, sec-pentyl acetate, 3-methoxybutyl acetate, methylpentyl acetate, 2-ethylbutyl acetate, 2-ethylhexyl acetate, benzyl acetate, cyclohexyl acetate, methylcyclohexyl acetate, n-nonyl acetate, and the like; a polyhydric alcohol-containing ether carboxylate-based solvent, such as ethylene glycol monomethyl ether acetate, ethylene glycol monoethyl ether acetate, diethylene glycol monomethyl ether acetate, diethylene glycol monoethyl ether acetate, diethylene glycol mono-n-butyl ether acetate, propylene glycol monomethyl ether acetate, propylene glycol monoethyl ether acetate, propylene glycol monopropyl ether acetate, propylene glycol monobutyl ether acetate, dipropylene glycol monomethyl ether acetate, dipropylene glycol monoethyl ether acetate, and the like; a lactone-based solvent, such as γ-butylolactone, δ-valerolactone, and the like; a carbonate-based solvent, such as dimethyl carbonate, diethyl carbonate, ethylene carbonate, propylene carbonate, and the like; a lactate ester-based solvent, such as methyl lactate, ethyl lactate, n-butyl lactate, n-amyl lactate, and the like; glycoldiacetate, methoxytriglycol acetate, propionic acid ethyl, propionic acid n-butyl, propionic acid isoamyl, diethyloxalate, di-n-butyloxalate, methyl acetoacetate, ethyl acetoacetate, malonic acid diethyl, phthalic acid dimethyl, phthalic diethyl, and the like.

Examples of the sulfoxide-based solvent are dimethyl sulfoxide, diethyl sulfoxide, and the like.

Examples of the hydrocarbon-based solvent are: an aliphatic hydrocarbon-based solvent, such as n-pentane, isopentane, n-hexane, isohexane, n-heptane, isoheptane, 2,2,4-trimethyl pentane, n-octane, isooctane, cyclohexane, methylcyclohexane, and the like; an aromatic hydrocarbon-based solvent, such as benzene, toluene, xylene, mesitylene, ethyl benzene, trimethyl benzene, methylethyl benzene, n-propyl benzene, isopropyl benzene, diethyl benzene, isobutyl benzene, triethyl benzene, diisopropyl benzene, n-amylnaphthalene, and the like.

In an embodiment, the organic solvent may be selected from an alcohol-based solvent, an amide-based solvent, an ester-based solvent, a sulfoxide-based solvent, and any combination thereof. In one or more embodiments, the organic solvent may be selected from propylene glycol monomethyl ether, propylene glycol monoethyl ether, propylene glycol monomethyl ether acetate, N-methyl-2-pyrrolidone, N,N-dimethylacetamide, ethyl lactate, dimethyl sulfoxide, and any combination thereof.

In an embodiment, when an acetal-type acid labile group is used, the organic solvent may further include alcohol having a high boiling point, such as diethylene glycol, propylene glycol, glycerol, 1,4-butanediol, or 1,3-butanediol, to accelerate a deprotection reaction of acetal.

The organic solvent may be used in an amount in a range of about 200 parts by weight to about 5,000 parts by weight, for example, about 400 parts by weight to about 3,000 parts by weight, based on 100 parts by weight of the base resin.

### Base Resin

The base resin may include a repeating unit represented by Formula 4 and including an acid labile group:

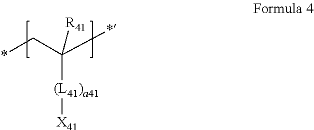

In Formula 4,
R₄₁ may be hydrogen, deuterium, a halogen, a linear or branched C₁-C₃₀ alkyl group, or a linear or branched C₁-C₃₀ halogenated alkyl group,
L₄₁ may be a single bond, O, C(═O), C(═O)O, OC(═O), C(═O)NH, NHC(═O), a substituted or unsubstituted C₁-C₃₀ alkylene group, a substituted or unsubstituted C₃-C₃₀ cycloalkylene group, a substituted or unsubstituted C₃-C₃₀ heterocycloalkylene group, a substituted or unsubstituted C₂-C₃₀ alkenylene group, a substituted or unsubstituted C₃-C₃₀ cycloalkenylene group, a substituted or unsubstituted C₃-C₃₀ heterocycloalkenyl group, a substituted or unsubstituted C₆-C₃₀ arylene group, or substituted or unsubstituted C₁-C₃₀ heteroarylene group,
a41 may be an integer from 1 to 6,
X₄₁ may be an acid labile group, and
* and *′ each indicate a binding site to a neighboring atom.

For example, R₄₁ in Formula 4 may be hydrogen, deuterium, a halogen, CH₃, CH₂F, CHF₂, or CF₃.

In Formula 4, L₄₁ may be selected from a single bond; C(═O); C(═O)O; OC(═O); C(═O)NH; NHC(═O); and a C₁-C₃₀ alkylene group, a C₃-C₃₀ cycloalkylene group, a C₃-C₃₀ heterocycloalkylene group, a C₂-C₃₀ alkenylene group, a C₃-C₃₀ cycloalkenylene group, a C₃-C₃₀ heterocycloalkenylene group, a C₆-C₃₀ arylene group, and a C₁-C₃₀ heteroarylene group, each unsubstituted or substituted with deuterium, a halogen, a cyano group, a hydroxy group, an amino group, a carboxylic acid group, an ester moiety, a sulfonic ester moiety, a carbonate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₃-C₂₀ cycloalkoxy group, a C₆-C₂₀ aryl group, or any combination thereof.

In Formula 4, a41 indicates the number of repetitions of L₁₁, wherein, when a41 is 2 or more, a plurality of L₁₁ may be identical to or different from each other.

In an embodiment, X₄₁ in Formula 4 may be represented by any one of Formulae 6-1 to 6-7:

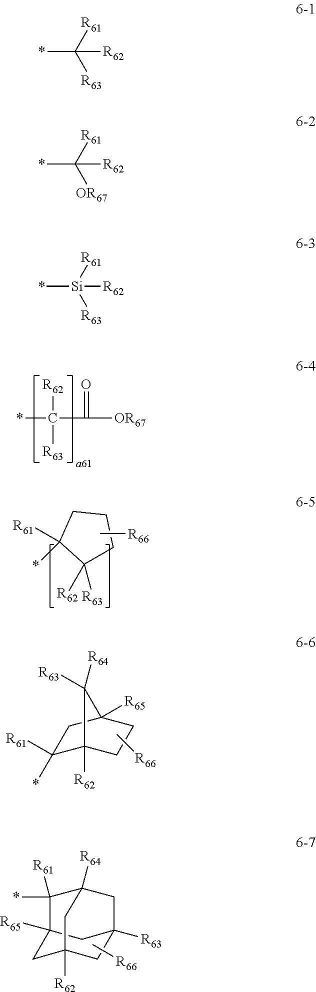

In Formulae 6-1 to 6-7,
a61 may be an integer from 0 to 6,
R₆₁ to R₆₆ may each independently be selected from hydrogen, deuterium, a halogen, a cyano group, a hydroxy group, an amino group, a carboxylic acid group, and a linear, branched, or cyclic C₁-C₂₀ hydrocarbon group that optionally includes a heteroatom,
R₆₇ may be a linear, branched, or cyclic C₁-C₂₀ monovalent hydrocarbon group that optionally includes a heteroatom,
two adjacent of R₆₁ to R₆₇ may optionally be bonded to each other to form a ring, and
* indicates a binding site to a neighboring atom.

When a61 in Formulae 6-4 and 6-5 is 0, (CR₆₂R₆₃)ₐ₆₁ may be a single bond.

In an embodiment, the repeating unit represented by Formula 4 may be represented by one of Formulae 4-1 and 4-2:

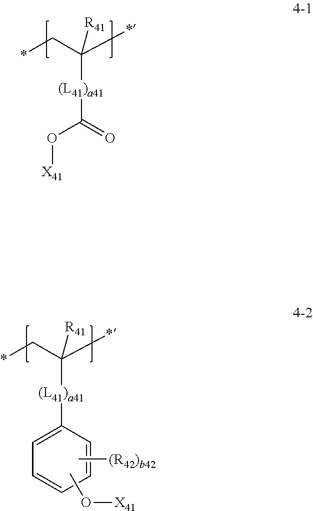

In Formulae 4-1 and 4-2,
L₄₁ and X₄₁ are each defined the same as described above,
a41 may be an integer from 1 to 4,
R₄₂ may be hydrogen, or a linear, branched, or cyclic C₁-C₁₀ monovalent group that optionally includes a heteroatom,
b42 may be an integer from 1 to 4, and
* and *′ each indicate a binding site to a neighboring atom.

The base resin including the repeating unit represented by Formula 4 may be decomposed under the action of an acid to generate a carboxyl group, and thus may be converted to have alkali solubility.

The base resin may further include, in addition to the repeating unit represented by Formula 4, a repeating unit represented by Formula 5:

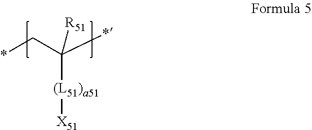

In Formula 5,
R₅₁ may be hydrogen, deuterium, a halogen, a linear or branched C₁-C₃₀ alkyl group, or a linear or branched C₁-C₃₀ halogenated alkyl group,
L₅₁ may be a single bond, O, C(═O), C(═O)O, OC(═O), C(═O)NH, NHC(═O), a substituted or unsubstituted C₁-C₃₀ alkylene group, a substituted or unsubstituted C₃-C₃₀ cycloalkylene group, a substituted or unsubstituted C₃-C₃₀ heterocycloalkylene group, a substituted or unsubstituted C₂-C₃₀ alkenylene group, a substituted or unsubstituted C₃-C₃₀ cycloalkenylene group, a substituted or unsubstituted C₃-C₃₀ heterocycloalkenyl group, a substituted or unsubstituted C₆-C₃₀ arylene group, or substituted or unsubstituted C₁-C₃₀ heteroarylene group,
a51 may be an integer from 1 to 6,
X₅₁ may be a non-acid labile group, and
* and *′ each indicate a binding site to a neighboring atom.

For example, R₅₁ in Formula 5 is defined the same as R₄₁ in Formula 4.

L₅₁ in Formula 5 is defined the same as L₄₁ in Formula 4.

In Formula 5, a51 indicates the number of repetitions of L₅₁, wherein, when a51 is 2 or more, a plurality of L₅₁ may be identical to or different from each other.

In an embodiment, X₅₁ in Formula 5 may be hydrogen, or a linear, branched, or cyclic C₁-C₂₀ monovalent hydrocarbon group that includes at least one polar moiety selected from a hydroxy group, a halogen, a cyano group, a carbonyl group, a carboxyl group, O, C(═O)O, OC(═O), S(═O)O, OS(═O), a lactone ring, a sultone ring, and a carboxylic acid anhydride moiety.

In an embodiment, the repeating unit represented by Formula 5 may be represented by any one of Formulae 5-1 and 5-2:

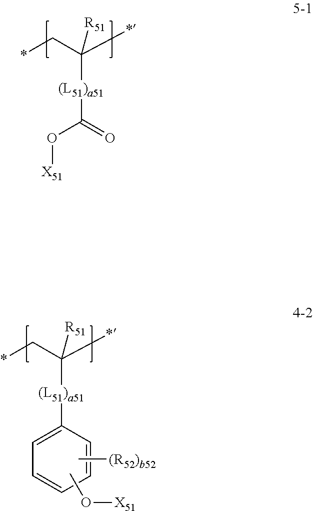

wherein, in Formulae 5-1 and 5-2,
L₅₁ and X₅₁ are each defined the same as described above,
a51 may be an integer from 1 to 4,
R₅₂ may be hydrogen or a hydroxyl group, or a linear, branched, or cyclic C₁-C₂₀ monovalent hydrocarbon group that optionally includes a heteroatom,
b52 may be an integer from 1 to 4, and
* and *′ each indicate a binding site to a neighboring atom.

For example, in an ArF lithography process, X₅₁ may include a lactone ring as a polar moiety, and in KrF, EB, and EUV lithography processes, X₅₁ may be phenol.

In an embodiment, the base resin may further include a moiety including an anion and/or a cation. For example, the base resin may further include a moiety in which a photoacid generator and/or a quencher are induced to bind to the side chain.

The base resin may have a weight average molecular weight (Mw) in a range of 1,000 to 500,000, for example, 3,000 to 100,000, wherein the weight average molecular weight (Mw) is measured by gel permeation chromatography using a tetrahydrofuran solvent and polystyrene as a standard material.

The base resin may have polydispersity index (PDI, Mw/Mn) in a range of 1.0 to 3.0, for example, 1.0 to 2.0. When the PDI of the base resin is satisfied within the ranges above, there is a less chance of remaining foreign substances on a pattern, or deterioration of a pattern profile may be minimized. Accordingly, the photoresist composition may become more suitable for forming a fine pattern.

The base resin may be prepared by any suitable method. For example, the base resin may be prepared by dissolving monomer(s) including unsaturated linking groups in an organic solvent, followed by performing thermal polymerization in the presence of a radical initiator.

In the base resin, a mole fraction (mol %) of each repeating unit derived from each monomer is as follows, but is not limited thereto:
i) the repeating unit represented by Formula 4 is included in an amount in a range of 1 mol % to 60 mol %, for example, 5 mol % to 50 mol %, and for example, 10 mol % to 50 mol %; and
ii) the repeating unit represented by Formula 5 is included in an amount in a range of 40 mol % to 99 mol %, for example, 50 mol % to 95 mol %, and for example, 50 mol % to 90 mol %.

The base resin may be a homopolymer, or may include a mixture of two or more types of polymers having a different composition, weight average molecular weight (Mw), and/or PDI (Mw/Mn).

### Photoacid Generator

The carboxylate salt may act as a photoacid generator since an acid is generated when decomposed by exposure, and thus, the photoresist composition may not include a separate photoacid generator.

However, when the carboxylate salt represented by Formula 1 acts as a quencher, the carboxylate salt may be used in combination with a photoacid generator that generates an acid.

The photoacid generator may be any compound capable of generating an acid when exposed to high energy rays such as UV rays, DUV rays, EB rays, EUV rays, X-rays, α-rays, γ-rays, and the like.

The photoacid generator may include a sulfonium salt, an iodonium salt, or a combination thereof.

In an embodiment, the photoacid generator may be represented by Formula 7:

B₇₁⁺A₇₁⁻ Formula 7
wherein, in Formula 7,
B₇₁⁺ may be represented by Formula 7A, and A₇₁⁻ may be represented by one of Formulae 7B to 7D, and
B₇₁⁺ and A₇₁⁻ may optionally be linked via a carbon-carbon covalent bond:

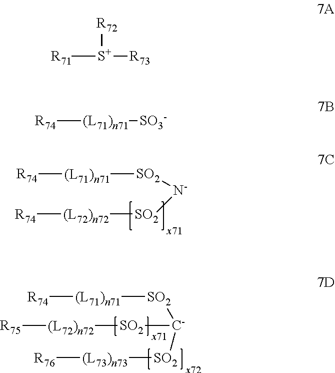

In Formulae 7A to 7D,
L₇₁ to L₇₃ may each independently be a single bond or CRR′,
R and R′ may each independently be hydrogen, deuterium, a halogen, a cyano group, a hydroxyl group, a C₁-C₂₀ alkyl group, a C₁-C₂₀ halogenated alkyl group, a C₁-C₂₀ alkoxy group, a C₃-C₂₀ cycloalkyl group, or a C₃-C₂₀ cycloalkoxy group,
n71 to n73 may each independently be 1, 2, or 3,
x71 and x72 may each independently be 0 or 1,
R₇₁ to R₇₃ may each independently be a linear, branched, or cyclic C₁-C₂₀ monovalent hydrocarbon group,
an adjacent two of R₇₁ to R₇₃ may optionally be bonded to each other to form a ring, and
R₇₄ to R₇₆ may each independently be: hydrogen; halogen; or a linear, branched, or cyclic C₁-C₂₀ monovalent hydrocarbon group that optionally includes a heteroatom.

For example, in Formula 7, B₇₁⁺ may be represented by Formula 7A, and A₇₁⁻ may be represented by Formula 7B. In detail, R₇₁ to R₇₃ in Formula 7 may each be a phenyl group.

The photoacid generator may be included in an amount in a range of 0 parts by weight to 40 parts by weight, 0.1 parts by weight to 40 parts by weight, or 0.1 parts by weight to 20 parts by weight, based on 100 parts by weight of the base resin. When the amount of the photoacid generator is satisfied within the ranges above, proper resolution may be achieved, and problems related to foreign particles after development or during stripping may be reduced.

One type of the photoacid generator may be used, or a combination of two or more different types of the photoacid generator may be used.

### Quencher

The quencher may be a salt that generates an acid having a weaker acidity than the acid generated from the carboxylate salt represented by Formula 1 and/or the photoacid generator.

The quencher may include a sulfonium salt, an iodonium salt, and a combination thereof.

In an embodiment, the quencher may be represented by Formula 8:

B₈₁⁺A₈₁⁻ Formula 8

In Formula 8,
B₈₁⁺ may be represented by any one of Formulae 8A to 8C, and A₈₁⁻ may be represented by any one of Formulae 8D to 8F, and
B₈₁⁺ and A₈₁⁻ may optionally be linked via a carbon-carbon covalent bond,

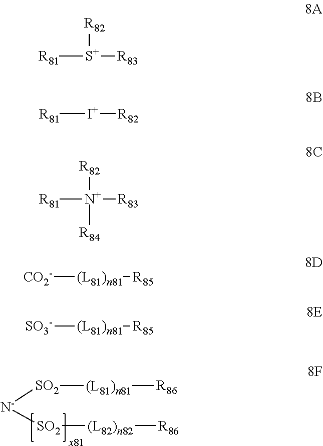

In Formulae 8A to 8F,
L₈₁ and L₈₂ may each independently be a single bond or CRR′,
R and R′ may each independently be hydrogen, deuterium, a halogen, a cyano group, a hydroxyl group, a C₁-C₂₀ alkyl group, a C₁-C₂₀ halogenated alkyl group, a C₁-C₂₀ alkoxy group, a C₃-C₂₀ cycloalkyl group, or a C₃-C₂₀ cycloalkoxy group,
n81 and n82 may each independently be 1, 2, or 3,
x81 may be 0 or 1,
R₈₁ to R₈₄ may each independently be a linear, branched, or cyclic C₁-C₂₀ monovalent hydrocarbon group,
an adjacent two of R₈₁ to R₈₄ may optionally be linked to each other to form a ring, and
R₈₅ and R₈₆ may each independently be: hydrogen; a halogen; or a linear, branched, or cyclic C₁-C₂₀ monovalent hydrocarbon group that optionally includes a heteroatom.

The quencher may be included in an amount of 0.01 parts by weight to 10 parts by weight, 0.05 parts by weight to 5 parts by weight, or 0.1 parts by weight to 3 parts by weight, based on 100 parts by weight of the base resin. When the amount of the photoacid generator is satisfied within the ranges above, proper resolution may be achieved, and problems related to foreign particles after development or during stripping may be reduced.

One type of the quencher may be used, or two or more different types thereof may be used in combination.

### Optional Components

The photoresist composition may further include a surfactant, a cross-linking agent, a leveling agent, a colorant, or any combination thereof, as needed.

The photoresist composition may further include a surfactant to improve coating properties, developability, and the like. Examples of the surfactant are: a non-ionic surfactant, such as polyoxyethylene lauryl ether, polyoxyethylene stearyl ether, polyoxyethylene oleyl ether, polyoxyethylene n-octylphenyl ether, polyoxyethylene n-nonylphenyl ether, polyethylene glycol dilaurate, polyethylene glycol distearate, and the like; and the like. For use as the surfactant, a commercially available product may be used, or a synthetic product may be used. Examples of the commercially available product are KP341 (manufactured by Shin-Etsu Chemical Co., Ltd.), POLYFLOW No. 75 and POLYFLOW No. 95 (manufactured by Kyoeisha Chemical Co., Ltd.), FTOP EF301, FTOP EF303, and FTOP EF352 (manufactured by Mitsubishi Materials Electronic Chemicals Co., Ltd.), MEGAFACE F171 (registered trademark), MEGAFACE F173, R40, R41, and R43 (manufactured by DIC Corporation), Fluorad FC430 (registered trademark) and Fluorad FC431 (manufactured by 3M Company), AsahiGuard AG710 (product of AGC Corporation), Surflon S-382 (registered trademark), Surflon SC-101, Surflon SC-102, Surflon SC-103, Surflon SC-104, Surflon SC-105, and Surflon SC-106 (manufactured by AGC Seimi Chemical Co., Ltd.), and the like.

The surfactant may be included in an amount in a range of about 0 parts by weight to about 20 parts by weight based on 100 parts by weight of the base resin. One type of the surfactant may be used, or a combination of two or more different types of the surfactant may be used.

A method of preparing the photoresist composition is not particularly limited. For example, a method of mixing the carboxylate salt, the base resin, the photoacid generator, and optional components added as necessary in the organic solvent may be used. The temperature or time at the mixing is not particularly limited. Filtration may be performed after the mixing as needed.

### Pattern Forming Method

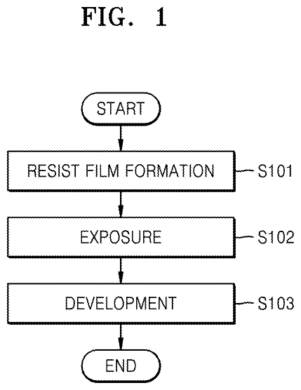

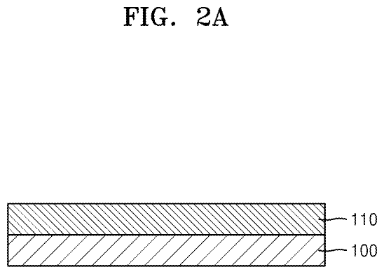

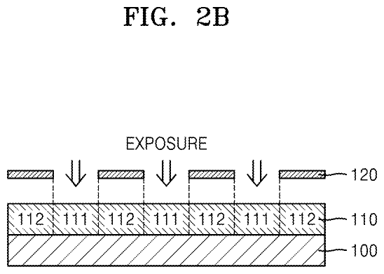

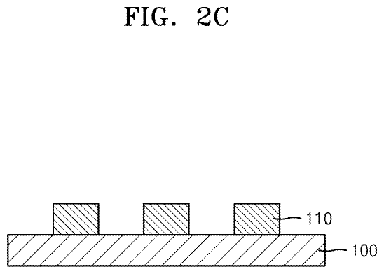

Hereinafter, a method of forming a pattern according to an embodiment will be described in detail with reference to FIGS. 1 and 2A to 2C. FIG. 1 is a flowchart representing a pattern forming method according to an embodiment, and FIGS. 2A to 2C are side cross-sectional views illustrating a pattern forming method according to an embodiment. Hereinafter, a method of forming a pattern by using a positive photoresist composition will be described in detail as an example, but embodiments are not limited thereto.

Referring to FIG. 1, a method of forming a pattern includes: forming a photoresist film by applying a photoresist composition (S101); exposing at least a portion of the photoresist film with high-energy rays (S102); and developing the exposed photoresist film by using a developing solution (S103). The operations above may be omitted as necessary, or may be performed in different orders.

First, referring to FIG. 2A, a board 100 is prepared. The board 100 may be, for example, a semiconductor board, such as a silicon board or a germanium board, or may be formed of glass, quartz, ceramic, copper, and the like. In an embodiment, the board 100 may include a Group III-V compound, such as GaP, GaAs, GaSb, and the like.

A photoresist film 110 may be formed by coating the board 100 with a photoresist composition to a desired thickness according to a coating method. As needed, a heating process may be performed thereon to remove an organic solvent remaining in the photoresist film 110. The coating method may include spin coating, dipping, roller coating, or other common coating methods. Among these methods, spin coating may be particularly used, and the photoresist film 110 having a desired thickness may be formed by adjusting the viscosity, concentration, and/or spin speed of the photoresist composition. In an embodiment, a thickness of the photoresist film 110 may be in a range of about 10 nm to about 300 nm. In one or more embodiments, a thickness of the photoresist film 110 may be in a range of about 30 nm to about 200 nm.

The lower limit of a pre-baking temperature may be 60° C. or higher, for example, 80° C. or higher. In addition, the upper limit of a pre-baking temperature may be 150° C. or lower, for example, 140° C. or lower. The lower limit of a pre-baking time may be 5 seconds or more, for example, 10 seconds or more. The upper limit of a pre-baking time may be 600 seconds or less, for example, 300 seconds or less.

Before coating the board 100 with the photoresist composition, a film to be etched (not shown) may be further formed on the board 100. The film to be etched may refer to a layer in which an image is transferred from a photoresist pattern and converted into a desired and/or alternatively predetermined pattern. In an embodiment, the film to be etched may be formed to include, for example, an insulating material, such as silicon oxide, silicon nitride, or silicon oxynitride. In one or more embodiments, the film to be etched may be formed to include a conductive material, such as metal, metal nitride, metal silicide, or metal silicide nitride. In one or more embodiments, the film to be etched may be formed to include a semiconductor material, such as polysilicon.

In an embodiment, an antireflection layer may be further formed on the board 100 to exhibit efficiency of the photoresist at most. The antireflection layer may be an organic-based antireflection layer or an inorganic-based antireflection layer.

In an embodiment, a protective layer may be further provided on the photoresist film 110 to reduce the influence of alkaline impurities included in the process. In addition, when performing immersion exposure, for example, a protective film against immersion may be provided on the photoresist film 110 to avoid direct contact between an immersion medium and the photoresist film 110.

Next, referring to FIG. 2B, at least a portion of the photoresist film 110 may be exposed with high-energy rays. For example, high-energy rays passing through a mask 120 may be irradiated to at least a portion of the photoresist film 110. Accordingly, the photoresist film 110 may have an exposed area 111 and an unexposed area 112.

The exposure may be carried out by irradiating high-energy rays through a mask having a desired and/or alternatively predetermined pattern and by using liquid, such as water or the like, as a medium in some cases. Examples of the high-energy rays are electromagnetic waves, such as ultraviolet rays, far-ultraviolet rays, extreme ultraviolet rays (EUV rays, wavelength of 13.5 nm), X-rays, γ-rays, and the like; charged particle beams, such as electron beams (EBs), α rays, and the like; and the like. The irradiation of such high-energy rays may be collectively referred to as “exposure.”

For use as a light source of the exposure, various types of irradiation including irradiating laser beams in the ultraviolet region, such as KrF excimer laser (wavelength of 248 nm), ArF excimer laser (wavelength of 193 nm), and an F₂ excimer laser (wavelength of 157 nm), irradiating harmonic laser beams in the far ultraviolet or vacuum ultraviolet region by a wavelength conversion method using laser beams from a solid-state laser source (e.g., YAG or semiconductor laser), irradiating electron beams or EUV rays, or the like may be used. Upon the exposure, the exposure may be performed through a mask corresponding to a desired pattern. However, when the light source of the exposure is electron beams, the exposure may be performed by direct drawing without using a mask.

The integral dose of the high-energy rays may be less than or equal to 2,000 mJ/cm², for example, less than or equal to 500 mJ/cm², in the case of using EUV rays as the high-energy rays. In addition, in the case of using electron beams as the high-energy rays, the integral dose of the high-energy rays may 5,000 μC/cm² or less, for example, 1,000 μC/cm² or less.

In addition, post-exposure baking (PEB) may be performed after the exposure. The lower limit of a PEB temperature may be 50° C. or higher, for example, 80° C. or higher. The upper limit of the PEB temperature may be 180° C. or less, for example, 130° C. or less. The lower limit of a PEB time may be 5 seconds or more, for example, 10 seconds or more. The upper limit of a PEB time may be 600 seconds or less, for example, 300 seconds or less.

Next, referring to FIG. 2C, the exposed photoresist film 110 may be developed by using a developing solution. The exposed area 111 may be washed away by the developing solution, whereas the unexposed area 112 may remain without being washed away by the developing solution.

For use as the developing solution, an alkali developing solution, a developing solution containing an organic solvent (hereinafter also referred to as “organic developing solution”), and the like may be used. As a developing method, a dipping method, a puddle method, a spray method, a dynamic administration method, and the like may be used. The developing temperature may be, for example, 5° C. or more and 60° C. or less, and the developing time may be, for example, 5 seconds or more and 300 seconds or less.

The alkali developing solution may be, for example, an alkaline aqueous solution which dissolves at least one alkaline compound, such as sodium hydroxide, potassium hydroxide, sodium carbonate, sodium silicate, sodium metasilicate, aqueous ammonia, ethylamine, n-propylamine, diethylamine, di-n-propylamine, triethylamine, methyldiethylamine, ethyldimethylamine, triethanolamine, tetramethyl ammonium hydroxide (TMAH), pyrrole, piperidine, choline, 1,8-diazabicyclo[5.4.0]-7-undecene (DBU), 1,5-diazabicyclo[4.3.0]-5-nonene, and the like. The alkaline developing solution may further include a surfactant.

The lower limit of the amount of the alkaline compound in the alkaline developing solution may be 0.1 mass % or more, for example, 0.5 mass % or more, and for example, 1 mass % or more. In addition, the upper limit of the amount of the alkaline compound in the alkaline developing solution may be 20 mass % or less, for example, 10 mass % or less, and for example, 5 mass % or less.

After the development, a resulting photoresist pattern may be washed with ultrapure water, and subsequently, the water remaining on the board 100 and the pattern may be removed.

As the organic solvent included in the organic developing solution, for example, the same organic solvent as an example in the <Organic solvent> section of the [Photoresist Composition] may be used.

The lower limit of the amount of the organic solvent in the organic developing solution may be 80 mass % or more, for example, 90 mass % or more, and for example, 95 mass % or more, and for example, 99 mass % or more.

The organic developing solution may also include a surfactant. In addition, the organic developing solution may include a trace amount of moisture. In addition, upon the development, the solvent may be substituted with a solvent of a different kind from the organic developing solution to stop the development.

The photoresist pattern resulting from the development may be further washed. As a washing solution, ultrapure water, a liquid rinse, and the like may be used. The liquid rinse is not particularly limited as long as it does not dissolve the photoresist pattern, and a solution containing a general organic solvent may be used. For example, the liquid rinse may be an alcohol-based solvent or an ester-based solvent. After the washing, the liquid rinse remaining on the board and the pattern may be removed. In addition, when ultrapure water is used, the water remaining on the board and the pattern may be removed.

In addition, the developing solution may be used either individually or in a combination of two or more.

After the photoresist pattern is formed as described above, an etching process may be performed thereon to obtain a patterned wiring board. The etching method may be performed by known methods including: dry etching using plasma gas; wet etching using an alkali solution, a cupric chloride solution, a ferric chloride solution; and the like.

After the photoresist pattern is formed, plating may be performed. Although not particularly limited, the plating method may include, for example, copper plating, solder plating, nickel plating, gold plating, and the like.

The photoresist pattern remaining after the etching may be exfoliated with an organic solvent. Although not particularly limited, examples of the organic solvent are propylene glycol monomethyl ether acetate (PGMEA) propylene glycol monomethyl ether (PGME), ethyl lactate (EL), and the like. Although not particularly limited, examples of the exfoliation method are an immersion method, a spray method, and the like. In addition, the wiring board on which the photoresist pattern is formed may be a multilayer wiring board, and may have through holes with a small diameter.

In an embodiment, after the formation of the photoresist pattern, the wiring board may be formed by a so-called lift-off process in which metal is deposited in a vacuum and then the photoresist pattern is dissolved in a solution.

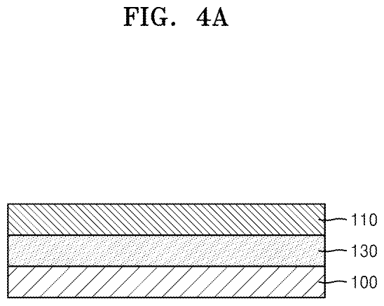

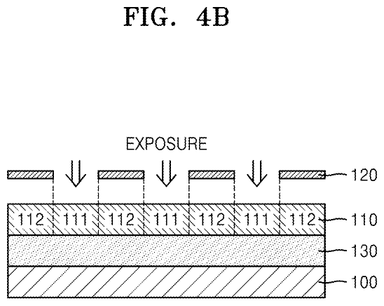

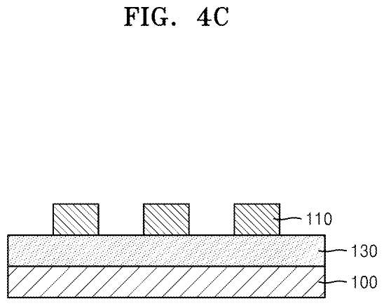

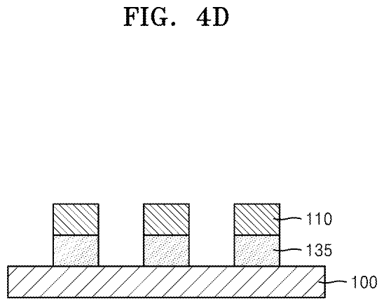

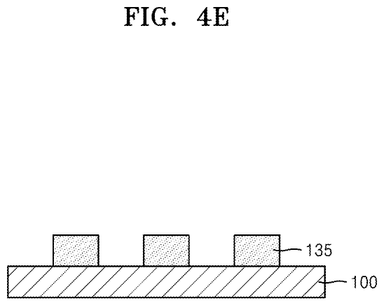

FIGS. 4A to 4E are side cross-sectional views illustrated a method of forming a patterned structure according to an embodiment. The method in FIGS. 4A to 4E may be the same as the method in FIGS. 2A to 2C, except for the differences described below.

Referring to FIG. 4A, a material layer 130 may be formed on the board 100 before forming a photoresist film 110 on the board 100. The photoresist film 110 may be formed on top of the material layer 130. The material layer 130 may include an insulating material (e.g., silicon oxide, silicon nitride), a semiconductor material (e.g., silicon), a metal (e.g., copper). In some embodiments, the material layer 130 may be a multi-layer structure. A material of the material layer 130 may be different than a material of the board 100.

Referring to FIG. 4B, like the operations described in FIGS. 2A to 2B, the photoresist film 110 may undergo a pre-exposure bake process and may be exposed with high energy rays through a mask 120, after which the photoresist film 110 may include exposed areas 111 and unexposed areas 112.

Referring to FIG. 4C, like the operation described in FIG. 2C, the exposed photoresist film 110 may be developed by using a developing solution. The exposed area 111 may be washed away by the developing solution, whereas the unexposed area 112 may remain without being washed away by the developing solution.

Referring to FIG. 4D, exposed areas of the material layer 130 may be etched using the photoresist pattern 110 to form a material pattern 135 on the board 100.

Referring to FIG. 4E, the photoresist pattern 110 may be removed using an ashing process.

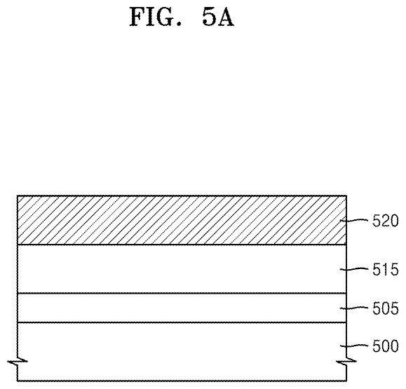

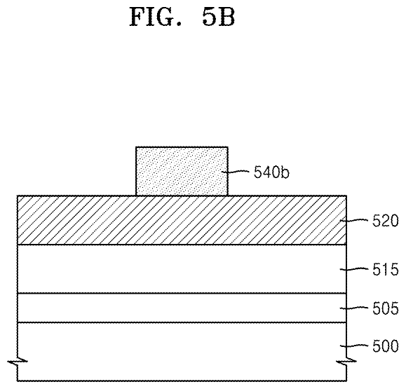

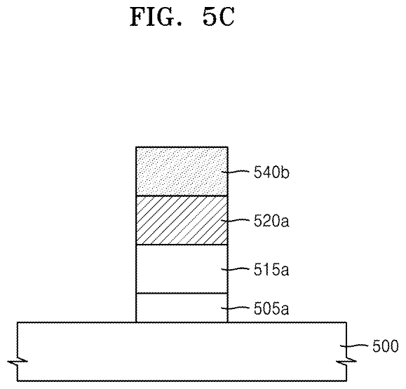

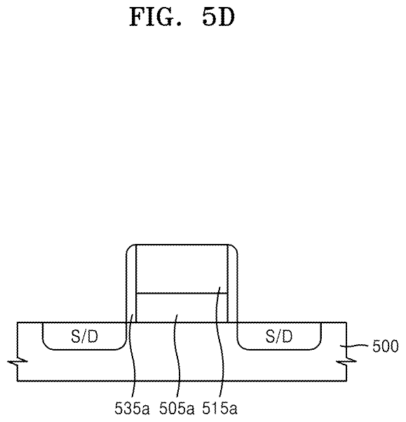

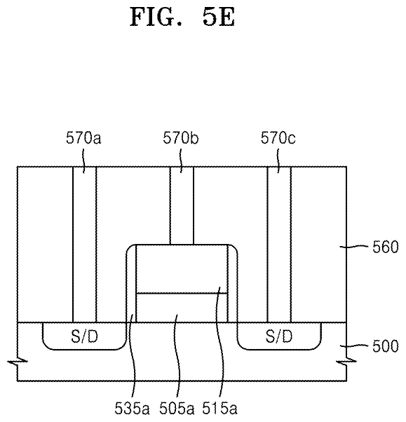

FIGS. 5A to 5E are side cross-sectional views illustrated a method of forming a semiconductor device according to an embodiment.

Referring to FIG. 5A, a gate dielectric 505 (e.g., silicon oxide) may be formed on a substrate 500. The substrate 500 may be a semiconductor substrate, such as a silicon substrate. A gate layer 515 (e.g., doped polysilicon) may be formed on the gate dielectric 505. A hardmask layer 520 may be formed on the gate layer 515.

Referring to FIG. 5B, a photoresist pattern 540b may be formed on the hardmask layer 520. The photoresist pattern 540b may be formed using a photoresist composition including a carboxylate salt according to example embodiments.

Referring to FIG. 5C, the gate layer 515 and the gate dielectric 505 may be etched to form a hardmask pattern 520, a gate electrode pattern 515a, and a gate dielectric pattern 505a.

Referring to FIG. 5D, a spacer layer may be formed over the gate electrode pattern 515a and the gate dielectric pattern 505a. The spacer layer may be formed using a deposition process (e.g., CVD). The spacer layer may be etched to form spacers 535a (e.g., silicon nitride) on sidewalls of the gate electrode pattern 515a and the gate dielectric pattern 505a. After forming the spacers 535a, ions may be implanted into the substrate 500 to form source/drain impurity regions S/D.

Referring to FIG. 5E, an interlayer insulating layer 560 (e.g., oxide) may be formed on the substrate 500 to cover the gate electrode pattern 515a, gate dielectric pattern 505a, and spacers 535a. Then, electrical contacts 570a, 570b, and 570c may be formed in the interlayer insulating layer 560 to connect to the gate electrode 515a and the S/D regions. The electrical contacts may be formed of a conductive material (e.g., metal). Although not illustrated, a barrier layer may be formed between sidewalls of the interlayer insulating layer 560 and the electrical contacts 570a, 570b, and 570c. While FIGS. 5A to 5E illustrate an example of forming a transistor, inventive concepts are not limited thereto. A photoresist composition including a carboxylate salt according one or more embodiments may be used in a patterning process to form other types of semiconductor devices.

The disclosure will be described in more detail with reference to Examples and Comparative Examples below, but the technical scope of the disclosure is not limited thereto.

### EXAMPLES

### Synthesis Example 1

### Synthesis of Compound A

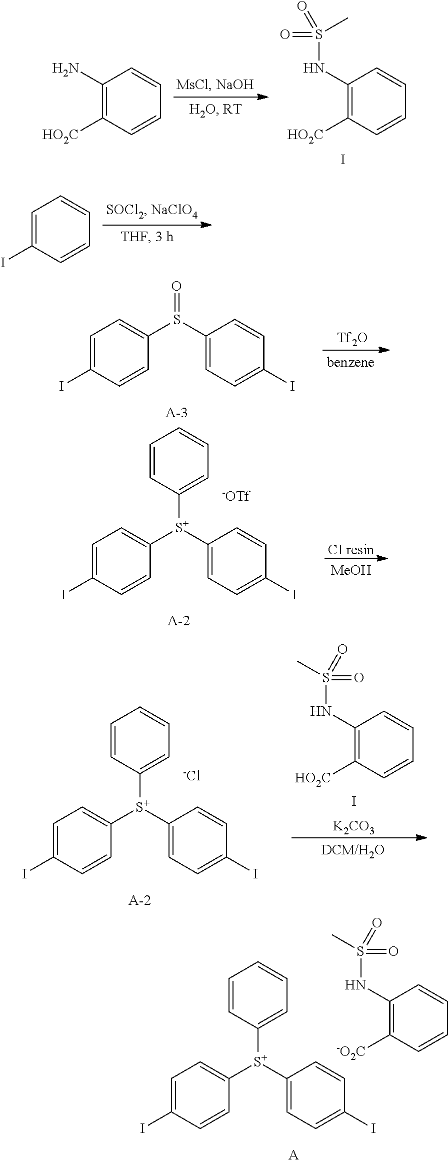

### Synthesis of Compound I

Anthranilic acid (5 g, 36.46 mmol) and sodium hydroxide (1.75 g, 43.75 mmol) were mixed with 243 mL of water to dissolve them all. Methanesulfonyl chloride (2.96 mL, 38.28 mmol) was slowly added to the solution being stirred, and a reaction proceeded for 20 hours. After the pH of the reaction solution was lowered to 2 or less by using a hydrogen chloride aqueous solution (12N), the mixture was stirred for 30 minutes and the resulting solid was separated by filtration. The solid thus separated was dissolved in 100 ml of ethyl acetate, followed by washing with 100 mL of water to obtain an organic layer, which was then dried with Na₂SO₄ and subjected to filtration. The filtrate was distilled under reduced pressure, and the resulting residue was recrystallized and purified with ethyl acetate and hexane to obtain Compound I (3.1 g, 40%). The resulting compound was confirmed by ¹H-NMR and LC-MS.

¹H-NMR (500 MHz, CD₂Cl₂) δ 10.3 (s, 1H), 8.14 (dd, 1H), 7.74 (d, 1H), 7.64 (ddd, 1H), 7.19 (ddd, 1H), 3.08 (s, 3H).

MS (ESI−) m/z 214.0216.

### Synthesis of Compound A-3

Iodobenzene (2.246 g, 11.01 mmol), thionyl chloride (0.655 g, 5.51 mmol), and sodium perchlorate (0.117 g, 1.10 mmol) were mixed with 12 mL of tetrahydrofuran, and stirred for 3 hours. Afterwards, the reaction solvent was removed by distillation under reduced pressure, and an organic layer obtained by extraction using 30 mL of water and 30 mL of dichloromethane was dried with Na₂SO₄ and filtered. The filtrate thus obtained was distilled under reduced pressure, and the resulting residue was separated and purified by silica gel column chromatography to obtain Compound A-3 (3.75 g, 75%). The resulting compound was confirmed by ¹H-NMR and LC-MS.

¹H-NMR (300 MHz, CDCl₃) δ 7.05 (d, 4H), 7.42 (d, 4H).

MS (M+H) m/z 454.85.

### Synthesis of Compound A-2

Compound A-3 (3.73 g, 8.20 mmol) was dissolved in 15 mL of benzene, and trifluoromethanesulfonic anhydride (2.778 g, 9.85 mmol) was added dropwise thereto at 0° C., and the resulting solution was stirred for 1 hour at room temperature. Next, an organic layer obtained by extraction using 20 mL of water and 50 mL of ethyl acetate was washed with a saturated NaHCO₃ aqueous solution, dried with MgSO₄, and then filtered. The filtrate thus obtained was distilled under reduced pressure, and the resulting residue was separated and purified by silica gel column chromatography to obtain Compound A-2 (4.92 g, 90%). The resulting compound was confirmed by ¹H-NMR and LC-MS.

¹H-NMR (300 MHz, CD₂Cl₂) δ 8.08 (d, 4H), 7.84 (t, 1H), 7.74 (t, 2H), 7.69 (d, 2H), 7.41 (d, 2H).

MS (cation) m/z 514.88.

### Synthesis of Compound A-1

Compound A-2 (2 g, 3.01 mmol) and Cl resin (10 g) were mixed with 10 mL of methanol, and stirred for 2 hours. Next, the resulting solution was filtered, and the filtrate was distilled under reduced pressure to obtain Compound A-1 (1.59 g, 96%). The resulting compound was confirmed by ¹H-NMR and LC-MS.

¹H-NMR (500 MHz, CD₂Cl₂) δ 8.05 (d, 4H), 7.82 (d, 2H), 7.78 (d, 1H), 7.70 (t, 2H), 7.61 (d, 4H).

MS (ESI+) m/z 514.8002.

### Synthesis of Compound A

Compound A-1 (0.5 g, 0.91 mmol), 2-(methylsulfonamido)benzoic acid (0.205 g, 0.953 mmol), and potassium carbonate (0.138 g, 0.999 mmol) were mixed with 5 mL of methylene chloride and 5 mL of water, and stirred for 2 hours. Next, an organic layer was isolated therefrom, and dried with MgSO₄, and then filtered. The filtrate was distilled under reduced pressure, and the resulting residue was washed with ether to obtain Compound A (0.63 g, 95%). The resulting compound was confirmed by ¹H-NMR and LC-MS.

¹H-NMR (500 MHz, CD₂Cl₂) δ 8.07-8.03 (m, 5H), 7.81-7.79 (m, 1H), 7.74-7.70 (m, 4H), 7.50-7.46 (m, 5H), 7.32 (ddd, 1H), 6.97 (ddd, 1H), 2.90 (s, 3H).

MS (ESI−) m/z 214.0342, MS (ESI+) m/z 515.8429.

### Evaluation Example 1

### Evaluation of Quenching Effect

A quenching effect was confirmed by the following method. 10 wt % of poly(4-vinylphenol) (Mw: about 11,000, purchase place: Sigma-Aldrich) and 6.5 wt % of Coumarin 6 (CAS No. 38215-36-0) were dissolved in to cyclohexanone, and then the respective photoacid generator and quencher shown in Table 1 were added thereto in the same mole number as Coumarin 6. A quartz plate having a size of 1 inch×1 inch was coated with the resulting solution to a thickness of 400 nm, and then dried at 130° C. for 2 minutes to form a thin film. After exposing the thin film to DUV (248 nm) at 10 mJ/cm² to 100 mJ/cm², the absorbance thereof was measured. Theoretically, since the absorbance of Coumarin 6 increases at a wavelength of 535 nm by acid generation, Coumarin 6 was used as an acid indicator. After assuming that Coumarin 6 was 100% converted for the absorbance intensity after the exposure at 100 mJ/cm², the absorbance intensity at each exposure dose was normalized to indicate the degree of acid generation.

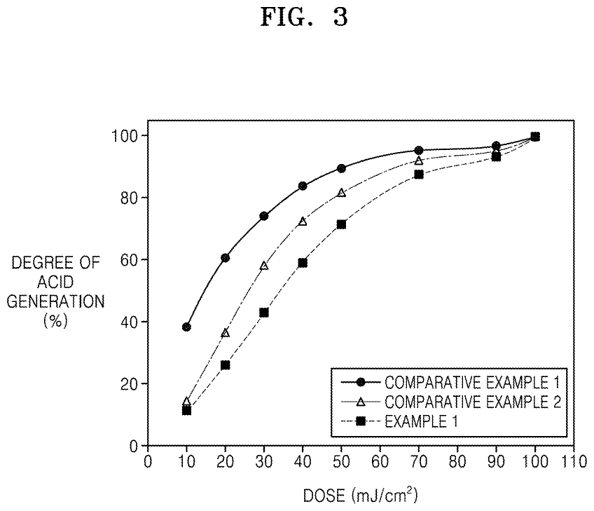

The degree of acid generation in a film formed using the polymer solution (Comparative Example 1) containing only the photoacid generator was compared with the absorbance of a film formed using the polymer solution (Comparative Example 2 and Example 1) containing the quencher together with the photoacid generator, and accordingly, a quenching effect, e.g., how much the quencher suppresses the acid generated by the photoacid generator, was measured, and results are shown in Table 1 and FIG. 3.

TABLE 1

| | | | Degree of | |
|---|---|---|---|---|
| | | | acid | |
| | | | generation | Quenching |
| | | | (%) | effect (%) |
| | Photoacid | | @ DUV | @ DUV |
| | generator | Quencher | 10 mJ | 10 mJ |
| Comparative Example 1 | 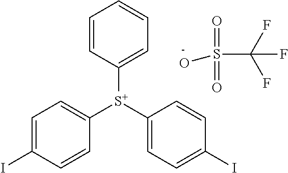 [SMILES: Ic1ccc([S+](c2ccccc2)c2ccc(I)cc2)cc1 ; O=S(=O)(O)C(F)(F)F] | — | 38.3 | — (Standard value) |
| Comparative Example 2 | 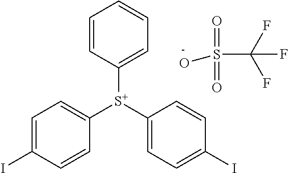 [SMILES: Ic1ccc([S+](c2ccccc2)c2ccc(I)cc2)cc1 ; O=S(=O)(O)C(F)(F)F] | 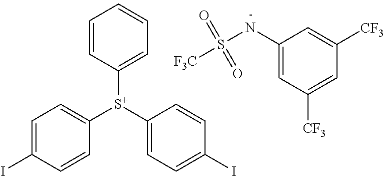 [SMILES: Ic1ccc([S+](c2ccccc2)c2ccc(I)cc2)cc1 ; O=S(=O)(Nc1cc(C(F)(F)F)cc(C(F)(F)F)c1)C(F)(F)F] | 15.2 | 60.4 |
| Example 1 | 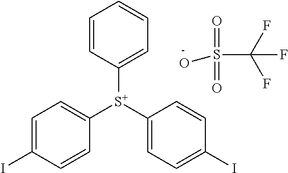 [SMILES: Ic1ccc([S+](c2ccccc2)c2ccc(I)cc2)cc1 ; O=S(=O)(O)C(F)(F)F] | 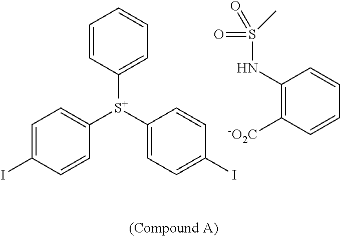 | 11.8 | 69.3 |

Referring to Table 1 and FIG. 3, it was confirmed that Comparative Example 2 shows a quenching effect of 60.4% at 10 mJ of DUV, whereas Example 1 shows an improved quenching effect by about 9% compared to Comparative Example 1.

The higher the quenching effect is, the more effectively the acid that penetrates into the non-exposed area can be removed, and thus the photoresist composition using the quencher of Example 1 seems to provide a pattern having improved resolution compared to the photoresist composition using the quencher of Comparative Example 2.

Embodiments of the present disclosure may provide: a carboxylate salt which may act as a photoacid generator capable of providing improved sensitivity and/or resolution and/or as a quencher having improved dispersibility and/or compatibility; and a photoresist composition including the same.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A carboxylate salt represented by Formula 1: 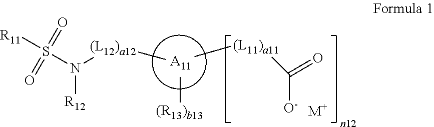
wherein, in Formula 1,
A₁₁ is a C₁-C₃₀ cyclic hydrocarbon group that optionally includes a heteroatom,
L₁₁ and L₁₂ are each independently a single bond or a divalent linking group,
a11 and a12 are each independently an integer from 0 to 4,
R₁₁ to R₁₃ are each independently hydrogen, deuterium, a halogen, a cyano group, a hydroxy group, a linear, branched, or cyclic C₁-C₂₀ monovalent hydrocarbon group that optionally includes a heteroatom, or —N(Q₁)(Q₂),
Q₁ and Q₂ are each independently hydrogen, deuterium, a linear, branched, or cyclic C₁-C₂₀ monovalent hydrocarbon group that optionally includes a heteroatom,
b13 is an integer from 0 to 10,
an adjacent two of R₁₁, R₁₂, R₁₃, L₁₁, and L₁₂ are optionally bonded to each other to form a ring,
an adjacent two of a plurality of R₁₃ are optionally bonded to each other to form a ring,
n11 and n12 are each independently an integer from 1 to 10, and
M⁺ is a counter cation.

2. The carboxylate salt of claim 1, wherein A₁₁ is a C₃-C₂₀ cycloalkyl group, a C₃-C₂₀ heterocycloalkyl group, a C₃-C₂₀ cycloalkenyl group, a C₃-C₂₀ heterocycloalkenyl group, a C₆-C₂₀ aryl group, or a C₁-C₂₀ heteroaryl group.

3. The carboxylate salt of claim 1, wherein L₁₁ and L₁₂ are each independently a single bond, O, C(═O), C(═O)O, OC(═O), C(═O)NH, NHC(═O), a linear, branched, or cyclic C₁-C₃₀ divalent hydrocarbon group that optionally includes a heteroatom.

4. The carboxylate salt of claim 1, wherein
a11 is 0.

5. The carboxylate salt of claim 1, wherein R₁₂ is hydrogen or deuterium.

6. The carboxylate salt of claim 1, wherein n11 and n12 are each independently 1 or 2.

7. The carboxylate salt of claim 1, wherein
M⁺ is a substituted or unsubstituted sulfonium cation, a substituted or unsubstituted iodonium cation, or a substituted or unsubstituted ammonium cation.

8. The carboxylate salt of claim 1, wherein M⁺ is represented by any one of Formulae 2-1 to 2-3: 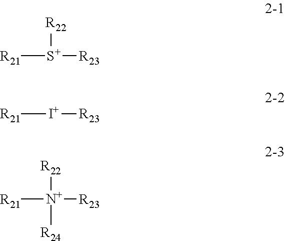
wherein, in Formulae 2-1 to 2-3,
R₂₁ to R₂₄ are each independently a linear, branched, or cyclic C₁-C₃₀ monovalent hydrocarbon group that optionally includes a heteroatom, and
an adjacent two of R₂₁ to R₂₄ are optionally bonded to each other to form a ring.

9. The carboxylate salt of claim 1, wherein M⁺ is represented by any one of Formulae 2-11 to 2-13: 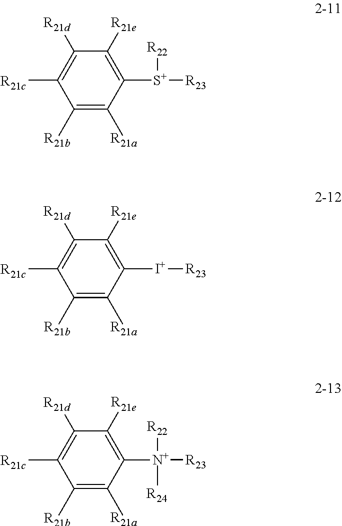
wherein, in Formulae 2-11 to 2-13,
R₂₁ₐ to R₂₁ₑ are each independently hydrogen, deuterium, a halogen, a cyano group, a hydroxy group, a linear, branched, or cyclic C₁-C₂₀ monovalent hydrocarbon group that optionally includes a heteroatom,
R₂₂ to R₂₄ are each independently a linear, branched, or cyclic C₁-C₃₀ monovalent hydrocarbon group that optionally includes a heteroatom, and
an adjacent two of R₂₁ₐ to R₂₁ₑ and R₂₂ to R₂₄ are optionally bonded to each other to form a ring.

10. The carboxylate salt of claim 1, wherein M⁺ is selected from any structure represented in Group II:

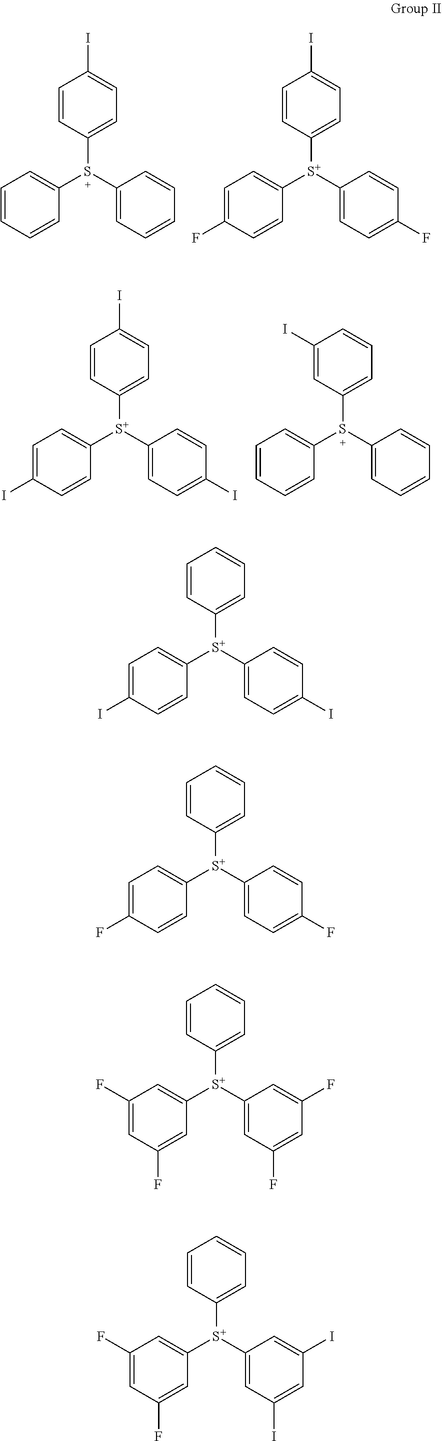

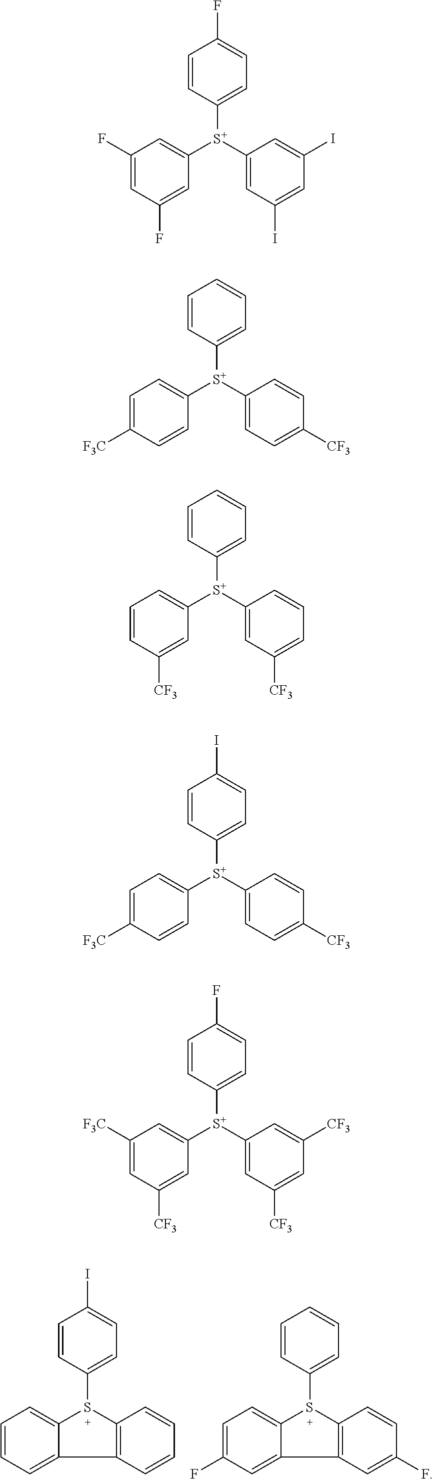

11. The carboxylate salt of claim 1, wherein
the carboxylate salt represented by Formula 1 is represented by one of Formulae 1-1 to 1-3: 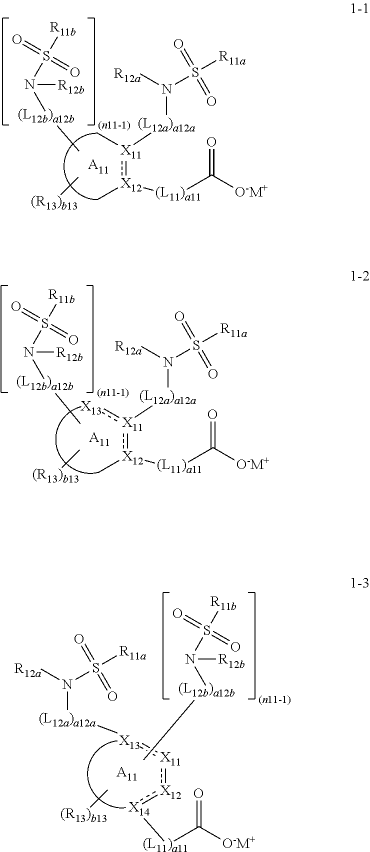
where, in Formulae 1-1 to 1-3,
X₁₁ to X₁₄ are each any atom,
the respective [symbol] indicates a single bond or a double bond,
A₁₁, L₁₁, a11, M⁺, R₁₃, n11, and b13 are defined the same as A₁₁, L₁₁, a11, M⁺, R₁₃, n11, and b13 in Formula 1,
R₁₁ₐ and R_{11b} are each defined the same as R₁₁ in Formula 1,
R₁₂ₐ and R_{12b} are each defined the same as R₁₂ in Formula 1,
L₁₂ₐ and L_{12b} are each defined the same as L₁₂ in Formula 1, and
a12a and a12b are each defined the same as a12 in Formula 1.

12. The carboxylate salt of claim 1, wherein the carboxylate salt represented by Formula 1 is represented by any one of Formulae 1-11 to 1-15: 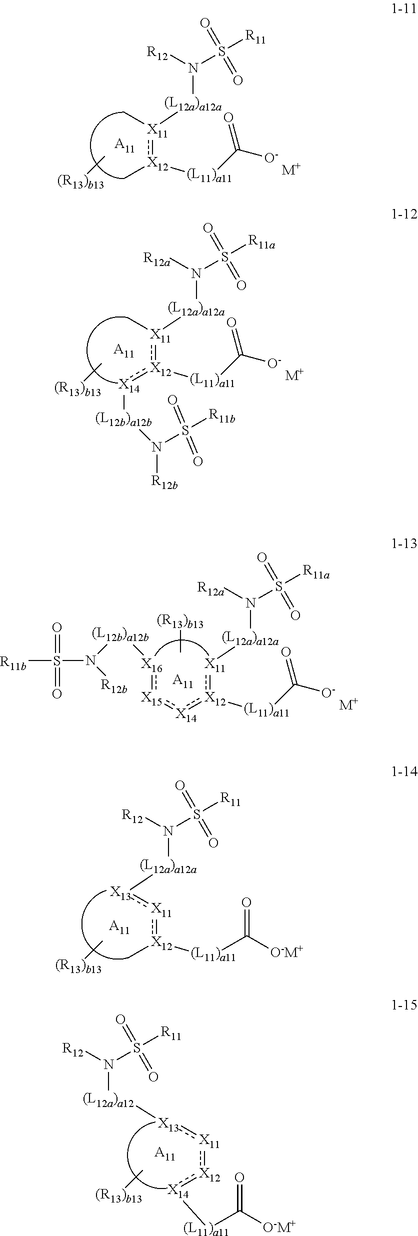
wherein, in Formulae 1-11 to 1-15,
X₁₁ to X₁₆ are each any atom,
the respective [symbol] indicates a single bond or a double bond,
A₁₁, L₁₁, L₁₂, a11, a12, M⁺, R₁₁ to R₁₃, n11, n12, and b13 are defined the same as A₁₁, L₁₁, L₁₂, a11, a12, M⁺, R₁₁ to R₁₃, n11, n12, and b13 in Formula 1,
R₁₁ₐ and R_{11b} are each defined the same as R₁₁ in Formula 1,
R₁₂ₐ and R_{12b} are each defined the same as R₁₂ in Formula 1,
L₁₂ₐ and L_{12b} are each defined the same as L₁₂ in Formula 1, and
a12a and a12b are each defined the same as a12 in Formula 1.

13. The carboxylate salt of claim 1, wherein the carboxylate salt represented by Formula 1 is selected from any salt represented in Group I: 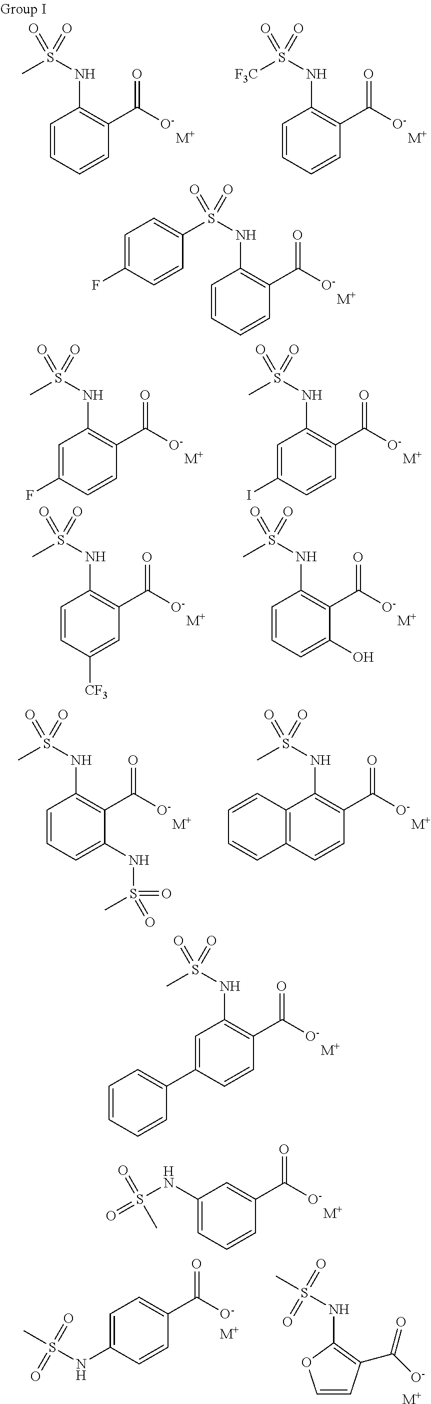 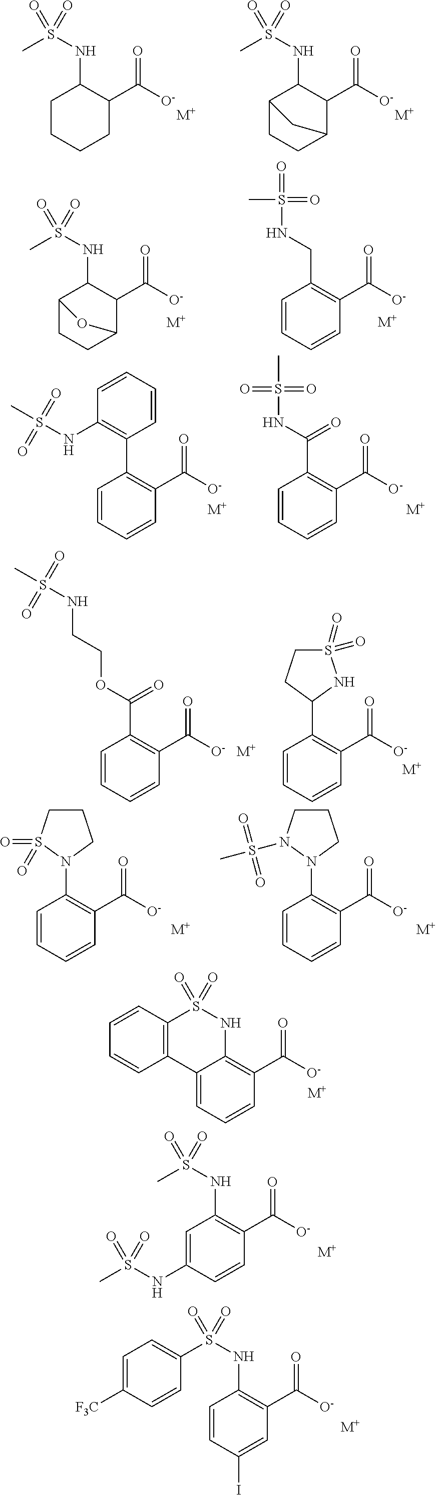 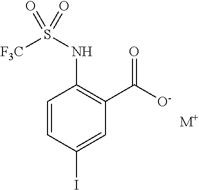
wherein, in Group I,
M⁺ is a counter cation.

14. A photoresist composition comprising:
the carboxylate salt of claim 1; an organic solvent; and
a base resin.

15. The photoresist composition of claim 14, further comprising:
a photoacid generator,
wherein the carboxylate salt is a photodegradable compound that is configured to generate an acid by being exposed to light exposure and act as a quenching base for neutralizing an acid before light exposure. 16 The photoresist composition of claim 14, wherein the carboxylate salt is a photodegradable compound that is configured to generate an acid by being exposed to light exposure.

17. The photoresist composition of claim 16, further comprising:
a quencher.

18. The photoresist composition of claim 14, wherein
an amount of the carboxylic acid is in a range of 0.1 parts by weight to 40 parts by weight based on 100 parts by weight of the base resin.

19. A method of forming a pattern, the method comprising:
forming a photoresist film by applying the photoresist composition of claim 14 on a substrate;
exposing at least a portion of the photoresist film to high energy rays to provide an exposed photoresist film; and
developing the exposed photoresist film using a developing solution.

20. The method of claim 19, wherein the exposing is performed by irradiating ultraviolet (UV) rays, deep ultraviolet (DUV) rays, extreme ultraviolet (EUV) rays, and/or electron beam (EB) rays.
